SEQUENCE LISTING (6 sequences)

SEQ ID NO: 1 - DNA, 3648 bp, homo sapiens
ggatcagctgactcgcctggctctgagccccgccgccgcgctcgggctccgtcagtttcctcggcagcggtaggcgagagcacgcggaggagcgtgcgcgggggccccgggagacggcggcggtggcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccatgtcccaaagtttactcaagactacccaggaacctcttgcccgagatcctgttaaacttcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattctccaaaacaattttctgcaggatgattgtacagaatcattgcttatgacatgatcgctttctacactgtattacataaataaattaaataaaataaccccgggcaagacttttctttgaaggatgactacagacattaaataatcgaagtaattttgggtggggagaagaggcagattcaattttctttaaccagtctgaagtttcatttatgatacaaaagaagatgaaaatggaagtggcaatataaggggatgaggaaggcatgcctggacaaacccttcttttaagatgtgtcttcaatttgtataaaatggtgttttcatgtaaataaatacattcttggaggagcaaaaaaaaaaaaaaaa

SEQ ID NO: 2 - protein, 770 aa, homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAMSQSLLKTTQEPLARDPVKLPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - DNA, 5811 bp, homo sapiens
ggacggccgagcggcagggcgctcgcgcgcgcccactagtggccggaggagaaggctcccgcggaggccgcgctgcccgccccctcccctggggaggctcgcgttcccgctgctcgcgcctgcgccgcccgccggcctcaggaacgcgccctcttcgccggcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggtgtcacagaggcagtggcagcaacaaaggatttgaaacttggtgtgttcgtggagccacaggcagacgatgtcaaccttgtgtgagtgtgacgggggttggggtggggcgggaggccacgggggaggccgaggcaggggctgggcagaggggagaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggtaaaaagagaaggcaagctggcaggagggtggcacttcgtggatgacctccttagaaaagactgaccttgatgtcttgagagcgctggcctcttcctccctccctgcagggtagggggcctgagttgaggggcttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattcccagcctcaccaccccttctcagtaatgaccctggttggttgcaggaggtacctactccatactgagggtgaaattaagggaaggcaaagtccaggcacaagagtgggaccccagcctctcactctcagttccactcatccaactgggaccctcaccacgaatctcatgatctgattcggttccctgtctcctcctcccgtcacagatgtgagccagggcactgctcagctgtgaccctaggtgtttctgccttgttgacatggagagagccctttcccctgagaaggcctggccccttcctgtgctgagcccacagcagcaggctgggtgtcttggttgtcagtggtggcaccaggatggaagggcaaggcacccagggcaggcccacagtcccgctgtcccccacttgcaccctagcttgtagctgccaacctcccagacagcccagcccgctgctcagctccacatgcatagtatcagccctccacacccgacaaaggggaacacacccccttggaaatggttcttttcccccagtcccagctggaagccatgctgtctgttctgctggagcagctgaacatatacatagatgttgccctgccctccccatctgcaccctgttgagttgtagttggatttgtctgtttatgcttggattcaccagagtgactatgatagtgaaaagaaaaaaaaaaaaaaaaaaggacgcatgtatcttgaaatgcttgtaaagaggtttctaacccaccctcacgaggtgtctctcacccccacactgggactcgtgtggcctgtgtggtgccaccctgctggggcctcccaagttttgaaaggctttcctcagcacctgggacccaacagagaccagcttctagcagctaaggaggccgttcagctgtgacgaaggcctgaagcacaggattaggactgaagcgatgatgtccccttccctacttccccttggggctccctgtgtcagggcacagactaggtcttgtggctggtctggcttgcggcgcgaggatggttctctctggtcatagcccgaagtctcatggcagtcccaaaggaggcttacaactcctgcatcacaagaaaaaggaagccactgccagctggggggatctgcagctcccagaagctccgtgagcctcagccacccctcagactgggttcctctccaagctcgccctctggaggggcagcgcagcctcccaccaagggccctgcgaccacagcagggattgggatgaattgcctgtcctggatctgctctagaggcccaagctgcctgcctgaggaaggatgacttgacaagtcaggagacactgttcccaaagccttgaccagagcacctcagcccgctgaccttgcacaaactccatctgctgccatgagaaaagggaagccgcctttgcaaaacattgctgcctaaagaaactcagcagcctcaggcccaattctgccacttctggtttgggtacagttaaaggcaaccctgagggacttggcagtagaaatccagggcctcccctggggctggcagcttcgtgtgcagctagagctttacctgaaaggaagtctctgggcccagaactctccaccaagagcctccctgccgttcgctgagtcccagcaattctcctaagttgaagggatctgagaaggagaaggaaatgtggggtagatttggtggtggttagagatatgcccccctcattactgccaacagtttcggctgcatttcttcacgcacctcggttcctcttcctgaagttcttgtgccctgctcttcagcaccatgggccttcttatacggaaggctctgggatctcccccttgtggggcaggctcttggggccagcctaagatcatggtttagggtgatcagtgctggcagataaattgaaaaggcacgctggcttgtgatcttaaatgaggacaatccccccagggctgggcactcctcccctcccctcacttctcccacctgcagagccagtgtccttgggtgggctagataggatatactgtatgccggctccttcaagctgctgactcactttatcaatagttccatttaaattgacttcagtggtgagactgtatcctgtttgctattgcttgttgtgctatggggggaggggggaggaatgtgtaagatagttaacatgggcaaagggagatcttggggtgcagcacttaaactgcctcgtaacccttttcatgatttcaaccacatttgctagagggagggagcagccacggagttagaggcccttggggtttctcttttccactgacaggctttcccaggcagctggctagttcattccctccccagccaggtgcaggcgtaggaatatggacatctggttgctttggcctgctgccctctttcaggggtcctaagcccacaatcatgcctccctaagaccttggcatccttccctctaagccgttggcacctctgtgccacctctcacactggctccagacacacagcctgtgcttttggagctgagatcactcgcttcaccctcctcatctttgttctccaagtaaagccacgaggtcggggcgagggcagaggtgatcacctgcgtgtcccatctacagacctgcagcttcataaaacttctgatttctcttcagctttgaaaagggttaccctgggcactggcctagagcctcacctcctaatagacttagccccatgagtttgccatgttgagcaggactatttctggcacttgcaagtcccatgatttcttcggtaattctgagggtggggggagggacatgaaatcatcttagcttagctttctgtctgtgaatgtctatatagtgtattgtgtgttttaacaaatgatttacactgactgttgctgtaaaagtgaatttggaaataaagttattactctgattaaa

SEQ ID NO: 4 - protein, 441 aa, homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - DNA, 3215 bp, homo sapiens
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 6 - protein, 140 aa, homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

(19) United States
(12) Patent Application Publication
(10) Pub. No.: US 2012/0141983 A1
(43) Pub. Date: Jun. 7, 2012

(54) Quantitative Aggregation Sensors

(76) Inventors: Alexandra Esteras Chopo, Stanford, CA (US); Isabella A. Graef, Woodside, CA (US)

(21) Appl. No.: 13/310,251

(22) Filed: Dec. 2, 2011

Related U.S. Application Data
(60) Provisional application No. 61/419,746, filed on Dec. 3, 2010.

(51) Int. Cl. C12Q 1/02; C07H 21/04; C12Q 1/68; C07K 19/00

(52) U.S. Cl. 435/6.1; 530/350; 536/23.4; 435/29

(57) ABSTRACT

Composition, systems and methods are provided for quantifying the amount of protein aggregation occurring in a cell in vitro and in vivo. These compositions, systems and methods find use in a number of applications, including in screening candidate agents for activity in modulating intracellular and extracellular protein aggregation in vitro and in vivo; in the generation of in vivo data for modeling aggregation processes in the cellular environment; for the validation of in vitro data, e.g. the effect of point mutations on the aggregation of amyloidogenic proteins; for the proteomic analysis of interacting partners so as to identify new therapeutic targets; for the analysis of changes in gene expression that are induced by intracellular versus extracellular aggregation; and for the evaluation of changes in the activity of the cellular network that controls protein folding and aggregation, the so-called proteostasis network.

### CROSS REFERENCE TO RELATED APPLICATIONS

Pursuant to 35 U.S.C. §119 (e), this application claims priority to the filing date of the U.S. Provisional Patent Application Ser. No. 61/419,746 filed Dec. 3, 2010; the disclosure of which is herein incorporated by reference.

### GOVERNMENT SUPPORT

This invention was made with Government support under contract EY016525 awarded by the National Institutes of Health. The Government has certain rights in this invention.

### BACKGROUND OF THE INVENTION

Oligomerization and deposition of protein aggregates play a central role in disease. A number of diseases share the pathological hallmark of intra- or extracellular insoluble proteins aggregates (Hinault, et al., 2006, Chaperones and proteases: cellular fold-controlling factors of proteins in neurodegenerative diseases and aging. J Mol Neurosci, 30(3): p. 249-65). The deposits may be systemic, such as accumulations of transthyretin in heart and liver as seen in senile systemic amyloidosis; or localized to a particular tissue, e.g. the brain as in the case of Alzheimer's (AD) or Parkinson's disease (PD). The proteins that are found in these aggregates vary; e.g. alpha-synuclein aggregates are associated with Parkinson's disease, islet amyloid polypeptide aggregates are associated with Type II Diabetes, and Aβ and tau polypeptide aggregates are associated with Alzheimer's Disease. Although some familial mutations can result in early onset of these diseases, aging is the major risk factor for many of them.

For example, Alzheimer's Disease (AD) is defined by the deposition of senile plaques, neurofibrillary tangles, and progressive neuronal loss in the brain of the patients (Selkoe, D. J. and M. B. Podlisny, 2002, Deciphering the genetic basis of Alzheimer's disease. Annu. Rev. Genomics Hum. Genet., 3:67-99). The process of extracellular plaque formation begins with the overproduction of the Alzheimer's disease peptide (Aβ), a 40- or 42-amino acid long peptide resulting from atypical cleavage of the amyloid precursor protein (APP). Aβ monomers are prone to adopt a misfolded conformation that can assemble into β-sheet enriched oligomers and protofibrils, which elongate to make up the insoluble fibrils mostly found deposited in amyloid senile plaques (Selkoe, D. J. and M. B. Podlisny, supra). Most of the Aβ deposits are found extracellularly, although intracellular aggregation has also been reported (Ohyagi, Y., 2008, Intracellular amyloid beta-protein as a therapeutic target for treating Alzheimer's disease. Curr Alzheimer Res 5(6):555-61). The major component of intracellular neurofibrillary tangles (NFT) in AD is Tau, a major neuronal microtubule-associated protein (Lee, V. M., 1996, Regulation of tau phosphorylation in Alzheimer's disease. Ann N Y Acad Sci 777:107-13). Its normal function is to promote and stabilize the assembly of microtubules from tubulin subunits. Hyperphosphorylated tau, particularly at sites in or adjacent to microtubule binding domains (e.g. Ser-262), has a reduced affinity for microtubules, and can enter the protein misfolding and oligomerization pathway, become toxic, and aggregate into paired helical filaments (PHFs). Tau pathology is also implicated in other neurodegenerative diseases known as tauopathies (Spires-Jones, T. L., et al., 2009, Tau pathophysiology in neurodegeneration: a tangled issue. Trends Neurosci, 32(3):150-9).

Extensive biophysical research and animal models are helpful to elucidate the different factors that govern amyloid formation. Quantitative in vitro and in vivo model of protein misfolding and aggregation is highly desirable. Such models will enable not only the screening of small molecule and genetic modulators of the aggregation process, but will provide exact measurements that can be used in modeling the aggregation process in a complex cellular environment. The present invention addresses these issues.

### SUMMARY OF THE INVENTION

Composition, systems and methods are provided for quantifying the amount of protein aggregation occurring in a cell in vitro and in vivo. These compositions, systems and methods find use in a number of applications, including screening candidate agents for activity in modulating intracellular and extracellular protein aggregation in vitro and in vivo; in the generation of in vivo data for modeling aggregation processes in the cellular environment; for the validation of in vitro data, e.g. the effect of point mutations on the aggregation of amyloidogenic proteins; for the proteomic analysis of interacting partners so as to identify new therapeutic targets; and for the analysis of changes in gene expression that are induced by intracellular versus extracellular aggregation; and for the evaluation of changes in the activity of the cellular network that controls protein folding and aggregation, the so-called proteostasis network.

In some aspects of the invention, an aggregation sensor is provided, the aggregation sensor comprising a reporter polypeptide fused to one or more aggregating peptides. In some embodiments, the aggregation sensor is an intracellular aggregation sensor and the reporter polypeptide is an intracellular polypeptide. In some embodiments, the aggregation sensor is an extracellular sensor and the reporter polypeptide is a secreted polypeptide.

In some embodiments, the reporter polypeptide is an enzyme, e.g. luciferase or β-galactosidase. In some embodiments, the reporter is a fluorescent polypeptide, e.g. GFP, RFP, dsRED, zFP506, zFP538, etc. In some embodiments, the aggregating peptide is an amyloidogenic peptide, e.g a peptide or polypeptide that is associated with the deposition of amyloids, e.g. an Aβ peptide, a Tau peptide, amylin, alpha-synuclein, etc. In some embodiments, the aggregating peptide is an Aβ peptide, a Tau peptide, or an α-synuclein peptide. In some embodiments, the Aβ peptide is Aβ40 or Aβ42. In some embodiments, the Aβ peptide is a variant of Aβ40 or Aβ42. In some embodiments, the variant of Aβ40 or Aβ42 comprises a substitution at a residue selected from the group consisting of residue 19, residue 20, and residue 22 of Aβ40 or Aβ42. In some embodiments, the Aβ substitution is F19P, F19D, F20E, or E22G. In some embodiments, the tau peptide is ₂₄₄Tau₃₇₂. In some embodiments, the α-synuclein peptide is an α-synuclein variant. In some embodiments, the variant comprises a substitution at residue 30. In certain embodiments, the substitution is A30P.

In some embodiments, the one or more aggregating peptides is fused to the N-terminus of the reporter polypeptide. In some embodiments, the one or more aggregating peptides is fused to the C-terminus of the reporter polypeptide. In some embodiments, the construct comprises two or more aggregating peptides, wherein one or more aggregating peptides is fused to the N-terminus of the reporter polypeptide and one or more aggregating peptides is fused to the C-terminus of the reporter polypeptide. In some embodiments, the construct further comprises a linker peptide inserted between the one or more aggregating peptides and the reporter polypeptide. In some embodiments, the linker is GGGGSGGGGS.

In some aspects of the invention, a nucleic acid encoding an aggregating sensor is provided. In some embodiments, the nucleic acid comprises an expression cassette encoding an aggregation sensor comprising a reporter polypeptide fused to one or more aggregating peptides. In some embodiments, the nucleic acid sequence encoding the aggregation sensor is operably linked to an inducible promoter. In some embodiments, the inducible promoter is a tetracycline promoter. In some embodiments, the nucleic acid is on a vector. In some embodiments, the vector is a plasmid. In some embodiments, the vector is a virus.

In some aspects of the invention, a cell that comprises an aggregating sensor or a nucleic acid encoding an aggregating sensor is provided. In some embodiments, the cell is in vitro. In some embodiments, the cell is in vivo. In some embodiments, the cell is a 293T cell. In some embodiments, the cell is a neuron. In some such embodiments, the neuron is a cortical neuron, a hippocampal neuron, or a dopaminergic neuron.

In some aspects of the invention, a method is provided for screening a candidate agent for activity in reducing the aggregation of polypeptides. In such methods, a cell comprising a aggregation sensor is contacted with the candidate agent. The activity of the reporter polypeptide of the aggregation sensor, e.g. the enzymatic activity or fluorescence activity of the reporter, is measured, and the measurement is compared to the measured activity of the reporter polypeptide of an aggregation sensor in a cell that was not contacted with the candidate agent. Greater activity of the reporter polypeptide in the cell that was contacted with the candidate agent as compared to in the cell that was not contacted with the agent indicates that the candidate agent has an activity in reducing the aggregation of polypeptides, e.g. polypeptides comprising the aggregating peptide. In some embodiments, the candidate agent is a small molecule, a nucleic acid, or a polypeptide. In some embodiments, the candidate agent that reduces the aggregation of polypeptides will treat a disease associated with aberrant amyloid formation. In some embodiments, the disease associated with aberrant amyloid formation is a neurodegenerative disease; Type 2 diabetes mellitus; medullary carcinoma of the thyroid; cardiac arrhythmias; solated atrial amyloidosis; atherosclerosis; rheumatoid arthritis; aortic medial amyloid; prolactinomas; familial amyloid polyneuropathy; hereditary non-neuropathic systemic amyloidosis; dialysis related amyloidosis; finnish amyloidosis; lattice corneal dystrophy; cerebral amyloid angiopathy; systemic AL amyloidosis; or Sporadic Inclusion Body Myositis. In some embodiments, the neurodegenerative disease is Alzheimer's disease, Parkinson's disease, transmissible spongiform encephalopathy, or Huntington's Disease.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1: Cartoon of sensor aggregation in the cytoplasmic (A) or secretory (B) compartment of mammalian cells. The cartoon also shows the effect of small molecule modulators or A-beta point mutations that interfere with protein aggregation and thus increase the activity of aggregation sensors.

FIG. 2: Schematic representation of fusion protein design: The transcription of all constructs was under control of the Actin promoter and all construct had a C-terminal HA-epitope tag. (A-C) Intracellular luciferase (cLuc) constructs. (A) Direct fusion of Aβ₄₂ to firefly luciferase. The N- and C-terminal direct fusion construct was only used for Aβ₄₂. (B) Fusion constructs containing a glycine linker between the enzyme and the insert (C) Split cLuciferase construct; (D) Secreted Luciferase (sLuc) linker fusion constructs; (E) Inserts fused to the reporters: peptide or protein domains and Aβ₄₂ variants. Aβ₄₂ variants were only used for cLuc and sLuc C-terminus linker fusions.

FIG. 3: Design and validation of aggregation sensors. (A) Schematic of aggregation sensor principle. (B) Cytoplasmic reporter activity and (C) protein expression of direct Aβ42 fusions to cLuc in the presence of DMSO or 10 μM lactacystin; blots were probed with cLuc, HA-tag, Aβ and actin antibodies. (D) Activity of N- or C-terminal fusions of Aβ42 or SH3 relative to untagged cLuc. (E) Relative activity of cLuc terminal fusions of Aβ42, its slower aggregation variantAβ40, a fragment of Tau244-372, α-synucleinA30P familial mutation compared to cLucSH3 in E15.5 murine cortical neurons. Data represents means±s.d. (F) Secreted reporter activity and (G) protein expression of Aβ42 and SH3 fusions to sLuc in the presence of DMSO or 5 μM lactacystin; blots were probed with HA-tag or Aβ antibodies. Luciferase activity was normalized on cotransfected β-Galactosidase (means±SD). Experiments were performed in transiently transfected 293T cells.

FIG. 4: Protein expression levels of the N- or C-terminal direct Aβ42 and SH3 fusions to cLuciferase assessed by Western-Blot. 293T cells were transiently transfected and the cells were lysed 40 hours after transfection. Blots were probed with antibodies against cLuciferase, HA and Aβ. Actin served as a loading control.

FIG. 5: Doxycycline induction of 293 FlpIn Trex cell lines expressing the secreted and cytoplasmic LucAβ and LucSH3 chimeric reporters. Cells were treated with doxycycline for 40 hours. (A) cLuc activity normalized by cell survival of cLucAβ and cLucSH3, (B) Western-Blot of cLucAβ and cLucSH3 using anti-HA or anti-Aβ antibodies. Actin serves as a loading control; (C) sLuc activity in the extracellular media normalized by cell survival of and sLucAβ and sLucSH3, (D) Western-Blot of extracellular sLucAβ and sLucSH3 using anti-HA or anti-Aβ antibodies

FIG. 6: In vitro kinetics of 25 μM synthetic Aβ42 monitored by (A) Thioflavin T (Tht) fluorescence (485 nm) and (B) Electron Microscopy; Scale bar: 500 nm (C) Schematic comparing the readout of the in vitro aggregation assay (ThT) to the cellular aggregation sensors. (D) Kinetics of cLucAβ enzymatic activity relative to cLucSH3, and (E) Immunostaining of Aβ or SH3 tagged cLuc 48 and 96 hours post-induction, arrows point to inclusion bodies formed by cLucAβ; Scale bars: 20 μm (48-96 (left) hrs); 5 μm (96 (right) hrs).(F) Quantification of inclusion body formation after 96 hours. (G) Kinetics of sLucAβ enzymatic activity relative to sLucSH3, and (H) filter trap assay of secreted aggregates larger than 0.22 μm. (I) Quantification of the filter trap assay.

FIG. 7: Kinetics of sLucAβ and sLucSH3 aggregation measured by filter trap assay. The figure shows three replicates per time point for each sample, corresponding to FIG. 6H. Cell culture medium was run as a negative control.

FIG. 8: Activity of the cytoplasmic (A) and the secreted (B) sensors fused to Aβvar compared to Aβ42 wt sensor activity in primary hippocampal neurons. (C) in vitro aggregation of synthetic Aβvar relative to Aβ42 wt measured by ThT fluorescence.

FIG. 9: Transient expression of cytoplasmic and secreted LucAβ and LucSH3 chimeric reporters in cultured primary hippocampal P0 neurons (A) cLuc activity and (B) sLuc activity 2 days after transfection; (C) Percentage of the signal of LucAβ compared to LucSH3 2 days and 4 days after cell transfection.

FIG. 10: Levels of protein expression by Western-Blot of Aβvariants and Aβ wt fused to (A) cLuciferase and (B) sLuciferase. 293T cells were transiently transfected, RIPA lysates (cLuc) and extracellular media were harvested 40 hours after transfection. Proteins were detected using antibodies against Aβ. Actin served as loading control.

FIG. 11: Mutations in the Aβ42 peptide that reduce Aβ42 aggregation in vitro (F19D, F19P) and in a Drosophila model (F20E) partially rescue loss of luciferase activity in cells transfected with intracellular luciferase constructs, whereas a mutation in the Aβ42 peptide related to a familial form of the disease (E22G) exacerbates the loss of luciferase activity typically observed of intracellular luciferase fused to wild type Aβ42 peptide. (A) Ratio of normalized Firefly activity of Luc-linker-Aβ42 mutants versus Luc-linker Aβ42. (B) Levels of protein expression.

FIG. 12: Mutations in the Aβ42 peptide that reduce Aβ42 aggregation in vitro (F19D, F19P) and in a Drosophila model (F20E) partially rescue loss of luciferase activity in cells transfected with secreted luciferase constructs, whereas a mutation in the Aβ42 peptide related to a familial form of the disease (E22G) exacerbates the loss of luciferase activity typically observed of intracellular luciferase fused to wild type Aβ42 peptide. Ratio of normalized luciferase activity of Luc-linker-Aβ42 mutants versus Luc-linker Aβ42.

FIG. 13: Expression of the intracellular aggregation sensor in primary murine neuronal cultures of E15.5 cortical neurons. (A) Activity of the intracellular luciferase sensor, normalized. (B) Ratio of luciferase activity in cells transfected with luciferase constructs comprising Aβ peptides comprising the F20E or E22G mutation versus in cells transfected with a luciferase construct comprising the wild type Aβ42 peptide.

FIG. 14: Expression of the secreted aggregation sensor in the extracellular fraction from primary murine neuronal cultures of E17.5 hippocampal neurons transfected by Amaxa. (A) Percentage of secreted luciferase activity in secLucAβ (“SLAB”)-transfected cells relative to secreted luciferase activity in secLucSH3 (“SLSH”)-transfected cells. (B) Ratio of secreted luciferase activity in cells transfected with the luciferase construct comprising the Aβ peptides comprising the F19P mutation versus in cells transfected with a luciferase construct comprising the wild type Aβ42 peptide.

FIG. 15: Activity of cytoplasmic (A) and secreted (B) reporters in the presence of 1 μM compounds compared to solvent alone. Data are mean±SE. **p<0.005, *p<0.05. (C) Inhibition of in vitro aggregation of synthetic Aβ42 (25 μM) by compounds (1 μM) relative to solvent alone measured by ThT fluorescence.

FIG. 16: Changes in proteostasis pathways 96 hours postinduction (A) relative mRNA levels of cells expressing cLucAβ or sLucAβ versus cLucSH3 or sLucSH3 measured by qPCR; (B) Protein expression analysed by Western blot. (C) Activity of cytoplasmic and secreted reporters normalized by cell survival in the presence of different concentrations of Thapsigargin compared to solvent alone. Data are means±s.d. Experiments were carried out using doxycyline inducible 293FlpInTrex cell lines expressing the aggregation sensors.

FIG. 17: Modeling Aβ aggregation in the living brain (A) Bioluminescence imaging of cLucAb and cLucSH3 in embryonic mouse brains. GFP fluorescence was used to localize the transfected area. ROI: region of interest (B) Quantification of photon flux in ROI normalized by GFP expression. (C) Western Blot analysis of brain lysates. (D) In vitro luciferase activity of brain lysates.

FIG. 18: Schematic representation of the generation of tetracycline-inducible (“tet”) transgenic ES cell lines or mice carrying an inducible aggregation sensor transgene. Transgene expression can be induced by treatment with doxycycline of cultured primary or differentiated cells.

### DETAILED DESCRIPTION OF THE INVENTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

Composition and methods are provided for quantifying the amount of protein aggregation occurring in a cell in vitro and in vivo. These compositions and methods find particular use in screening candidate agents for activity in modulating intracellular and extracellular protein aggregation in vitro and in vivo; in the generation of in vivo data for modeling aggregation processes in the cellular environment; for the validation of in vitro data, e.g. the effect of point mutations on the aggregation of amyloidogenic proteins; for the proteomic analysis of interacting partners so as to identify new therapeutic targets; and for the analysis of changes in gene expression that are induced by intracellular versus extracellular aggregation.

These and other objects, advantages, and features of the invention will become apparent to those persons skilled in the art upon reading the details of the compositions and methods as more fully described below.

A “DNA molecule” refers to the polymeric form of deoxyribonucleotides (adenine, guanine, thymine, or cytosine) in either single stranded form or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes.

A DNA “coding sequence” is a DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and synthetic DNA sequences. A polyadenylation signal and transcription termination sequence may be located 3′ to the coding sequence.

“DNA regulatory sequences”, as used herein, are transcriptional and translational control sequences, such as promoters, enhancers, polyadenylation signals, terminators, and the like, that provide for and/or regulate expression of a coding sequence in a host cell.

As used herein, a “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

As used herein, the term “reporter gene” refers to a coding sequence whose product, a “reporter polypeptide”, may be assayed easily and quantifiably when introduced into tissues or cells.

A “vector” is a replicon, such as plasmid, phage or cosmid, to which another DNA segment, i.e. an “insert”, may be attached so as to bring about the replication of the attached segment. A “construct” is a vector plus an insert.

An “expression cassette” comprises a protein coding sequence operably linked to a promoter. The promoter may be a constitutively active promoter, i.e. a promoter that is active in the absence externally applied agents, or it may be an inducible promoter, i.e. a promoter whose activity is regulated upon the application of an agent to the cell.

A “DNA construct” is a DNA molecule comprising a vector and an insert, e.g. an expression cassette.

A cell has been “transformed” or “transfected” by exogenous or heterologous DNA, e.g. a DNA construct, when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

The amino acids described herein are preferred to be in the “L” isomeric form. The amino acid sequences are given in one-letter code (A: alanine; C: cysteine; D: aspartic acid; E: glutamic acid; F: phenylalanine; G: glycine; H: histidine; I: isoleucine; K: lysine; L: leucine; M: methionine; N: asparagine; P: proline; Q: glutamine; R: arginine; S: serine; T: threonine; V: valine; W: tryptophan; Y: tyrosine; X: any residue). In keeping with standard polypeptide nomenclature, NH₂ refers to the free amino group present at the amino terminus (the N terminus) of a polypeptide, while COOH refers to the free carboxy group present at the carboxy terminus (the C terminus) of a polypeptide.

By a “reduced aggregation of a polypeptide”, it is meant that the amount of aggregation of a polypeptide in a cell is reduced by 1.5-fold or more, e.g. 2-fold or more, 3-fold or more, 4-fold or more, 8-fold or more 10-fold or more, 20-fold or more, 50-fold or more, 100-fold, or more, 500-fold or more, or 1000-fold or more as compared to the amount of aggregation of that reporter in a cell under control conditions.

By “increased aggregation of a polypeptide” it is meant that the amount of aggregation of that polypeptide in a cell is increased by 1.5-fold or more, e.g. 2-fold or more, 3-fold or more, 4-fold or more, 8-fold or more 10-fold or more, 20-fold or more, 50-fold or more, 100-fold, or more, 500-fold or more, or 1000-fold or more as compared to the amount of aggregation of that reporter in a cell under control conditions.

By “reduced activity of a reporter polypeptide” it is meant that the amount of enzymatic activity of that reporter polypeptide in a cell is reduced by 1.5-fold or more, e.g. 2-fold or more, 3-fold or more, 4-fold or more, 8-fold or more 10-fold or more, 20-fold or more, 50-fold or more, 100-fold, or more, 500-fold or more, or 1000-fold or more as compared to the enzymatic activity of that reporter in a cell under control conditions.

By “increased activity of a reporter polypeptide” it is meant that the amount of enzymatic activity of that reporter polypeptide in a cell is increased by about 1.5-fold or more, e.g. 2-fold or more, 3-fold or more, 4-fold or more, 8-fold or more 10-fold or more, 20-fold or more, 50-fold or more, 100-fold, or more, 500-fold or more, or 1000-fold or more as compared to the enzymatic activity of that reporter in a cell under control conditions.

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, and kits for genetic manipulation referred to in this disclosure are available from commercial vendors such as BioRad, Stratagene, Invitrogen, Sigma-Aldrich, and ClonTech.

Compositions, systems, and methods are provided for quantitatively assessing protein aggregation in vitro and in vivo. By protein aggregation it is meant the aggregation of mis-folded proteins, i.e. the nonspecific coalescence of misfolded proteins, believed to be caused by interactions between solvent-exposed hydrophobic surfaces that are normally buried within a protein's interior. Protein aggregation is thought to be responsible for many diseases such as neurodegenerative diseases (e.g., Alzheimer's disease, Parkinson's disease, transmissible spongiform encephalopathy, and Huntington's Disease); Type 2 diabetes mellitus; medullary carcinoma of the thyroid; cardiac arrhythmias; solated atrial amyloidosis; atherosclerosis; rheumatoid arthritis; aortic medial amyloid; prolactinomas; familial amyloid polyneuropathy; hereditary non-neuropathic systemic amyloidosis; dialysis related amyloidosis; finnish amyloidosis; lattice corneal dystrophy; cerebral amyloid angiopathy; systemic AL amyloidosis; or Sporadic Inclusion Body Myositis. Thus, the subject inventions have many uses, e.g. for screening candidate agents for activity in modulating intracellular and extracellular protein aggregation in vitro and in vivo; for generating in vivo data for modeling aggregation processes in the cellular environment; for validating in vitro data, e.g. the effect of point mutations on the aggregation of amyloidogenic proteins; for the proteomic analysis of interacting partners so as to identify new therapeutic targets; for the analysis of changes in gene expression that are induced by intracellular versus extracellular aggregation; and for the evaluation of changes in the activity of the cellular network that controls protein folding and aggregation , the so-called proteostasis network.

### Compositions

In some aspects of the invention, aggregation sensors are provided. Aggregation sensors are chimeric proteins comprising a reporter polypeptide fused to one or more aggregating peptides. By a reporter polypeptide it is meant a polypeptide having activity that may be easily and quantifiably measured. Any convenient reporter polypeptide may be used. For example, the reporter polypeptide may be a fluorescent protein whose fluorescence may be monitored, e.g. GFP, RFP, dsRED, zFP506, zFP538. As another example, the reporter polypeptide may be an enzyme whose activity may be monitored, e.g., by monitoring its effect on a substrate, e.g. b-galactosidase, luciferase, etc. The reporter polypeptide may be an intracellular protein, e.g. a nuclear protein, a cytoplasmic protein, a protein associated with cytoplasmic organelles or the cell membrane, etc., e.g. Firefly luciferase. Alternatively, the reporter polypeptide may be a secreted protein, e.g. an extracellular protein, e.g. Metridia luciferase.

In the subject aggregating sensors, the reporter polypeptide is fused to one or more aggregating peptides, e.g. 1, 2, 3, 4 or more aggregating peptides. An aggregating peptide is a peptide or polypeptide that promotes the aggregation of polypeptides to which they are fused. An aggregating peptide will increase the aggregation of a polypeptide by 1.5-fold or more, e.g. 2-fold or more, 3-fold or more, or 4-fold or more, sometimes 8-fold or more, 10-fold or more, 20-fold or more, or 50-fold or more, for example 10 0-fold, or more, 500-fold or more, or 1000-fold or more as compared to the amount of aggregation of that polypeptide in a cell under control conditions, e.g. when not fused to the aggregating peptide. Aggregating peptides may be readily identified by measuring the extent of polypeptide aggregation in the presence and absence of the peptide of interest. For example, the kinetics, or rate, of aggregation may be measured, e.g. by fusing the peptide to a reporter polypeptide and measuring the amount of time it takes for enzymatic activity of the reporter to develop and/or the rate of decay of that activity. As another example, the intracellular distribution of the polypeptide may be monitored, e.g. by fusing the peptide to a polypeptide and using histochemistry or epifluorescence microscopy to determine if and to what extent the polypeptide has concentrated in inclusion bodies. As a third example, the size of aggregates may be measured in the presence/absence of the peptide of interest, e.g. by filter trap assays.

In some embodiments, the aggregating peptide is associated with the formation amyloids, i.e. it is an amyloid, or “amyloidogenic”, peptide. In other words, it is a peptide that aggregates to form amyloid plaques. Amyloids are insoluble fibrous protein aggregates formed by the polymerization of polypeptide into cross-beta structures, e.g. a beta sheet structure. Abnormal accumulation of amyloid in organs has been associated with the progression of various diseases. A table of examples of such diseases/conditions and the amyloidogenic peptide associated with amyloid deposition in those diseases is provided below:

| Disease | Amyloid polypeptide | Symbol |
|---|---|---|
| Alzheimer's disease | Beta amyloid | APP or Aβ |
| Alzheimer's disease | Tau | ₂₄₄Tau₃₇₂ |
| Diabetes mellitus type 2 | IAPP (Amylin) | AIAPP |
| Parkinson's disease | α-synuclein | none |
| Transmissible | PrPSc | APrP |
| spongiform | | |
| encephalopathy e.g. | | |
| Bovine spongiform | | |
| encephalopathy | | |
| Huntington's Disease | Huntingtin | none |
| Medullary carcinoma | Calcitonin | ACal |
| of the thyroid | | |
| Cardiac arrhythmias, | Atrial natriuretic factor | AANF |
| Isolated atrial | | |
| amyloidosis | | |
| Atherosclerosis | Apolipoprotein AI | AApoA1 |
| Rheumatoid arthritis | Serum amyloid A | AA |
| Aortic medial amyloid | Medin | AMed |
| Prolactinomas | Prolactin | APro |
| Familial amyloid | Transthyretin | ATTR |
| polyneuropathy | | |
| Hereditary non- | Lysozyme | ALys |
| neuropathic systemic | | |
| amyloidosis | | |
| Dialysis related | Beta 2 microglobulin | Aβ2M |
| amyloidosis | | |
| Finnish amyloidosis | Gelsolin | AGel |
| Lattice corneal | Keratoepithelin | AKer |
| dystrophy | | |
| Cerebral amyloid | Beta amyloid | Aβ |
| angiopathy | | |
| Cerebral amyloid | Cystatin | ACys |
| angiopathy (Icelandic | | |
| type) | | |
| systemic AL amyloidosis | Immunoglobulin light | AL |
| | chain AL | |
| Sporadic Inclusion Body | S-IBM | none |
| Myositis | | |

Any polypeptide that promotes the aggregation of polypeptides, e.g. the amyloid polypeptides listed in the table above, may be used in the subject aggregation sensors. The aggregating peptide may be a native aggregating peptide or it may be a variant thereof. By “native aggregating peptide” it is meant a polypeptide found in nature. Using beta amyloid (“amyloid beta (A4) precursor protein” or “Aβ”, Genbank Accession No. NM_{—}000484.3, SEQ ID NO:1, encoding protein SEQ ID NO:2) as an example, native aggregating peptides would include any Aβ that naturally occurs in humans, as well as Aβ orthologs that naturally occur in other eukaryotes, e.g. protist, fungi, plants or animals, for example yeast, insects, nematodes, sponge, mammals, non-mammalian vertebrates. Using Tau (“MAPT”, Genbank Accession No. NM_{—}005910.5, SEQ ID NO: 3, encoding protein SEQ ID NO: 4) as another example, native aggregating peptides would include any Tau protein that naturally occurs in humans, as well as Tau orthologs that naturally occur in other eukaryotes, e.g. protist, fungi, plants or animals, for example yeast, insects, nematodes, sponge, mammals, non-mammalian vertebrates. Using α-synuclein (Genbank Accession No. NM_{—}000345.3, SEQ ID NO:5, encoding protein SEQ ID NO: 6) as a third example, native aggregating peptides would include any α-synuclein protein that naturally occurs in humans, as well as α-synuclein orthologs that naturally occur in other eukaryotes, e.g. protist, fungi, plants or animals, for example yeast, insects, nematodes, sponge, mammals, non-mammalian vertebrates. By “variant” it is meant a mutant of the native polypeptide having less than 100% sequence identity with the native sequence that still promotes polypeptide aggregation. Again using beta amyloid, tau, and α-synuclein as examples, variants would include polypeptides having 60% sequence identity or more with human Aβ (SEQ ID NO:2), or tau (SEQ ID NO:4), or α-synuclein (SEQ ID NO:6), e.g. 65%, 70%, 75%, or 80% or more identity, such as 85%, 90%, or 95% or more identity, for example, 98% or 99% identity with the full length native Aβ, Tau, or α-synuclein, respectively. Variants also include fragments of a native beta amyloid polypeptide that have aggregating activity, e.g. a fragment comprising residues 672-713 of Aβ (“Aβ₄₂”), or the comparable sequence in a beta amyloid homolog or ortholog), a fragment comprising residues 672-711 of Aβ (“Aβ₄₀”) or the comparable sequence in a beta amyloid homolog or ortholog), a fragment comprising residues 244-372 of Tau (“₂₄₄Tau₃₇₂”, or the comparable sequence in a beta amyloid homolog or ortholog), etc. Variants also include fragments that have aggregating activity and 60% sequence identity or more with a fragment of a native aggregating polypeptide, e.g. 65%, 70%, 75%, or 80% or more identity, such as 85%, 90%, or 95% or more sequence identity, for example, 98% or 99% identity with the comparable fragment of the native aggregating polypeptide, e.g. Aβ₄₂ F19D or Aβ₄₂ F19P, in which residue 19 of Aβ₄₂ has been mutated; Aβ₄₂ F20E, in which residue 20 of Aβ₄₂ has been mutated; Aβ₄₂ E22G, in which residue 22 of Aβ₄₂ has been mutated; α-synuclein A30P, in which residue 30 of α-synuclein has been mutated. It will be appreciated by the ordinarily skilled artisan that any aggregating peptide known in the art or as empirically determined may be employed.

The aggregating peptide may be fused to the reporter polypeptide at either terminus i.e. the N-terminus or C-terminus, of the reporter. In some instances, the aggregating peptide may be fused to the reporter polypeptide at both the N-terminus and the C-terminus. Alternatively, the aggregating peptide may be inserted within the sequence of the reporter polypeptide, e.g. at any convenient position within the reporter polypeptide that does not disrupt reporter polypeptide activity.

In some instances, the aggregating peptide is fused directly to the reporter polypeptide. In other instances, the aggregating peptide is separated from the reporter polypeptide by a linker, i.e. a stretch of 3-50 amino acids or more, e.g. 5-25 amino acids, 8-15 amino acids, or 10-12 amino acids, e.g. GGGGSGGGGS.

Typically, the aggregating sensor is provided to a cell as a nucleic acid that encodes the aggregating sensor as part of an expression cassette. That is, the expression cassette comprises nucleic acid sequence that encodes the aggregating sensor, i.e. nucleic acid sequence encoding a reporter polypeptide fused to one or more nucleic acid sequences encoding aggregating peptides. In some embodiments, the nucleic acid sequence encoding the aggregating sensor is operably linked to a constitutive active promoter, i.e. a promoter that is always active in a cell, e.g. the SV40 promoter, the HCMV promoter, etc. In other embodiments, the nucleic acid sequence encoding the aggregating sensor is operably linked to an inducible promoter, i.e. a promoter whose activity is regulated upon the application of an agent or a physical change to the cell, e.g. alcohol, tetracycline/doxycycline, steroids, metal, or other compounds, e.g. the TET-ON or TET-OFF promoter, the alcohol dehydrogenase promoter, glucocorticoid or estrogen receptor response elements, metallothionein promoters, etc, or a change in light or temperature, e.g. a light pulse or heat shock.

The expression cassette encoding the aggregating sensor may be placed on or in any vector suitable for the introduction of the expression cassette into the cell, e.g. plasmid, cosmid, minicircle, phage, virus, etc. depending on whether it is desirous to maintain the nucleic acid episomally e.g. as plasmids, minicircle DNAs, virus-derived vectors such cytomegalovirus, adenovirus, etc., or integrate the nucleic acid into the cell genome, e.g. through homologous recombination or random integration, e.g. retrovirus-derived vectors such as MMLV, HIV-1, ALV, etc.

### Host Cells

In aspects of the invention, methods are provided for screening candidate agents for activity in modulating intracellular and extracellular protein aggregation in vitro and in vivo; for generating in vivo data for modeling aggregation processes in the cellular environment; for validating in vitro data, e.g. the effect of point mutations on the aggregation of amyloidogenic proteins; for the proteomic analysis of interacting partners so as to identify new therapeutic targets; and for the analysis of changes in gene expression that are induced by intracellular versus extracellular aggregation. For these methods, a host cell expressing an aggregating sensor may be employed.

Cells useful for screening include any cell in which polypeptides comprising aggregating peptides are known to aggregate. Cells may be from established cell lines. e.g. 293T cells, CHO cells, NT2 cells, PC12 cells. They may be primary cells, where “primary cells”, “primary cell lines”, and “primary cultures” are used interchangeably herein to refer to cells and cells cultures that have been derived from a subject and allowed to grow in vitro for a limited number of passages, i.e. splittings, of the culture. For example, primary cultures are cultures that may have been passaged 0 times, 1 time, 2 times, 4 times, 5 times, 10 times, or times, but not enough times go through the crisis stage. Typically, the primary cell lines of the present invention are maintained for fewer than 10 passages in vitro. They may be cultures of induced differentiated cells, i.e. somatic cells induced from embryonic stem cells (ESCs), embryonic germ cells (EGs), induced pluripotent stem cells (iPSCs), fibroblasts, etc. by any method known in the art. Examples of somatic cells include any differentiated cells from ectodermal (e.g., neurons and fibroblasts), mesodermal (e.g., cardiomyocytes), or endodermal (e.g., pancreatic cells) lineages. The somatic cells may be one or more: pancreatic beta cells, neural stem cells, neurons (e.g., hippocampal neurons, cortical neurons, dopaminergic neurons), oligodendrocytes, oligodendrocyte progenitor cells, hepatocytes, hepatic stem cells, astrocytes, myocytes, hematopoietic cells, cardiomyocytes, and the like. They may be terminally differentiated cells, or they may be capable of giving rise to cells of a specific lineage, e.g. multipotent cell types such as neural stem cells, cardiac stem cells, or hepatic stem cells which may be differentiated into neurons, cardiomyocytes, or hepatocytes, respectively.

The subject cells may be isolated from fresh or frozen cells, which may be from a neonate, a juvenile or an adult, and from tissues including skin, nervous system, muscle, bone marrow, peripheral blood, umbilical cord blood, spleen, liver, pancreas, lung, intestine, stomach, and other differentiated tissues, e.g. by biopsy or aphoresis from a live donor, or obtained from a dead or dying donor within about 48 hours of death. For isolation of cells from tissue, an appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Nucleic acid encoding an aggregating sensor may be introduced into the host cell by any convenient method that promotes the cellular uptake of nucleic acid. For example, vectors may be provided directly to the subject cells. In other words, the host cells are contacted with vectors comprising the nucleic acid encoding the aggregating sensor such that the vectors are taken up by the cells. Methods for contacting cells with nucleic acid vectors, such as electroporation, calcium chloride transfection, and lipofection, are well known in the art.

Alternatively, the nucleic acid encoding the aggregating sensor may be provided to the subject cells via a virus. In other words, the host cells are contacted with viral particles comprising the nucleic acid expressing the aggregating sensor. Retroviruses, for example, lentiviruses, are particularly suitable to the method of the invention. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein to be incorporated into the capsid, this envelope protein determining the specificity of the viral particle for the cells. Envelope proteins are of at least three types, ecotropic, amphotropic and xenotropic. Retroviruses packaged with ecotropic envelope protein, e.g. MMLV, are capable of infecting most murine and rat cell types, and are generated by using ecotropic packaging cell lines such as BOSC23 (Pear et al. (1993) P.N.A.S. 90:8392-8396). Retroviruses bearing amphotropic envelope protein, e.g. 4070A (Danos et al, supra.), are capable of infecting most mammalian cell types, including human, dog and mouse, and are generated by using amphotropic packaging cell lines such as PA12 (Miller et al. (1985) Mol. Cell. Biol. 5:431-437); PA317 (Miller et al. (1986) Mol. Cell. Biol. 6:2895-2902); GRIP (Danos et al. (1988) PNAS 85:6460-6464). Retroviruses packaged with xenotropic envelope protein, e.g. AKR env, are capable of infecting most mammalian cell types, except murine cells. The appropriate packaging cell line may be used to ensure that the subject CD33+ differentiated somatic cells are targeted by the packaged viral particles. Methods of introducing the retroviral vectors comprising the nucleic acid expressing the aggregating sensor into packaging cell lines and of collecting the viral particles that are generated by the packaging lines are well known in the art.

After contacting the host-cells with the nucleic acid expressing the aggregating sensor, the contacted cells are cultured so as to select the outgrowth of cells comprising the nucleic acid expressing the aggregating sensor. In some instances, cells are selected for those that transiently maintain the nucleic acid. In other words, the nucleic acid does not integrate into the genome of the cell, but rather is maintained episomally. In other instances, cells are selected for those that stably maintain the nucleic acid, i.e. the nucleic acid integrates into the host genome and expresses the aggregating sensor. Methods for culturing cells such as those described above are well known in the art, any of which may be used in the present invention to grow, isolate and reculture the desired host cells expressing the aggregating sensor of choice.

### Utility

Aggregating sensors of the subject application are useful in a number of applications. For example, aggregating sensors may be used for screening candidate agents for activity in modulating intracellular and extracellular protein aggregation in vitro and in vivo; for generating in vivo data for modeling aggregation processes in the cellular environment; for validating in vitro data, e.g. the effect of point mutations on the aggregation of amyloidogenic proteins; for the proteomic analysis of interacting partners so as to identify new therapeutic targets; for the analysis of changes in gene expression that are induced by intracellular versus extracellular aggregation; and for the evaluation of changes in the activity of the cellular network that controls protein folding and aggregation, the so-called proteostasis network. Essentially, the compositions, systems, and methods disclosed herein may be used to better model diseases characterized by aberrant protein aggregation, and identify agents for preventing that protein aggregation, which, in turn, will be useful in treating the above-mentioned diseases and others.

For example, in assays to screen candidate agents for activity in modulating protein aggregation, host cells comprising the subject aggregation sensors, e.g. host cells as described above, are contacted with a candidate agent of interest and the effect of the candidate agent is assessed by monitoring one or more output parameters that are reflective of the extent of aggregation of the polypeptide encoded by the aggregation sensor construct. These output parameters are typically reflective of the activity of the reporter polypeptide encoded by aggregation sensor, e.g. luciferase activity, β-galactosidase activity, fluorescence activity, etc. Any convenient parameter that is reflective of the amount of reporter activity/reporter aggregation may be assessed. For example, the kinetics, or rate, of aggregation may be measured, e.g. by measuring the amount of time it takes for enzymatic activity of the reporter polypeptide to develop and/or the rate of decay of that activity. As another example, the intracellular distribution of the reporter polypeptide may be monitored, e.g. by fluorescence microscopy, e.g. to determine if and to what extent the reporter polypeptide has concentrated in inclusion bodies. As a third example, the size of aggregates may be measured, e.g. by filter trap assays. Other output parameters that may be measured include the number and size of protein inclusion bodies formed, e.g. by visualizing the subcellular location of gold particles. Alternatively or additionally, the output parameters may be reflective of the viability of the culture, e.g. the number of cells in the culture, the rate of proliferation of the culture. Alternatively or additionally, the output parameters may be reflective of the function of the cells in the culture, e.g. the cytokines and chemokines produced by the cells, the rate of chemotaxis of the cells, the cytotoxic activity of the cells, etc.

Parameters are quantifiable components of cells, particularly components that can be accurately measured, desirably in a high throughput system. A parameter can be any cell component or cell product including cell surface determinant, receptor, protein or conformational or posttranslational modification thereof, lipid, carbohydrate, organic or inorganic molecule, nucleic acid, e.g. mRNA, DNA, etc. or a portion derived from such a cell component or combinations thereof. While most parameters will provide a quantitative readout, in some instances a semi-quantitative or qualitative result will be acceptable. Readouts may include a single determined value, or may include mean, median value or the variance, etc. Characteristically a range of parameter readout values will be obtained for each parameter from a multiplicity of the same assays. Variability is expected and a range of values for each of the set of test parameters will be obtained using standard statistical methods with a common statistical method used to provide single values.

Candidate agents of interest for screening include known and unknown compounds that encompass numerous chemical classes, primarily organic molecules, which may include organometallic molecules, inorganic molecules, genetic sequences, etc. An important aspect of the invention is to evaluate candidate drugs, including toxicity testing; and the like. Candidate agents include organic molecules comprising functional groups necessary for structural interactions, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof. Included are pharmacologically active drugs, genetically active molecules, etc. Compounds of interest include chemotherapeutic agents, hormones or hormone antagonists, etc. Exemplary of pharmaceutical agents suitable for this invention are those described in, “The Pharmacological Basis of Therapeutics,” Goodman and Gilman, McGraw-Hill, New York, N.Y., (1996), Ninth edition. Also included are toxins, and biological and chemical warfare agents, for example see Somani, S. M. (Ed.), “Chemical Warfare Agents,” Academic Press, New York, 1992).

Candidate agents of interest for screening also include nucleic acids, for example, nucleic acids that encode siRNA, shRNA or antisense molecules, or nucleic acids that encode polypeptides. Many vectors useful for transferring nucleic acids into target cells are available. The vectors may be maintained episomally, e.g. as plasmids, minicircle DNAs, virus-derived vectors such cytomegalovirus, adenovirus, etc., or they may be integrated into the target cell genome, through homologous recombination or random integration, e.g. retrovirus derived vectors such as MMLV, HIV-1, ALV, etc.

Vectors may be provided directly to the subject cells. In other words, the host cells expressing the aggregation sensor are contacted with vectors comprising the nucleic acid of interest such that the vectors are taken up by the cells. Methods for contacting cells with nucleic acid vectors, such as electroporation, calcium chloride transfection, and lipofection, are well known in the art. Alternatively, the nucleic acid candidate agents may be provided to the subject cells via a virus, e.g. as described above for introducing the nucleic acid encoding an aggregation sensor to a host cell. Vectors used for providing nucleic acid candidate agents to the subject cells will typically comprise suitable promoters for driving the expression, that is, transcriptional activation, of the nucleic acid of interest. This may include ubiquitously acting promoters, for example, the CMV-b-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold. In addition, vectors used for providing reprogramming factors to the subject cells may include genes that must later be removed, e.g. using a recombinase system such as Cre/Lox, or the cells that express them destroyed, e.g. by including genes that allow selective toxicity such as herpesvirus TK, bcl-xs, etc

Candidate agents of interest for screening also include polypeptides. Such polypeptides may optionally be fused to a polypeptide domain that increases solubility of the product. The domain may be linked to the polypeptide through a defined protease cleavage site, e.g. a TEV sequence, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like.

If the candidate polypeptide agent is being assayed for its ability to inhibit aggregation signaling intracellularly, the polypeptide may comprise the polypeptide sequences of interest fused to a polypeptide permeant domain. A number of permeant domains are known in the art and may be used in the non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

If the candidate polypeptide agent is being assayed for its ability to inhibit aggregation signaling extracellularly, the polypeptide may be formulated for improved stability. For example, the peptides may be PEGylated, where the polyethyleneoxy group provides for enhanced lifetime in the blood stream. The polypeptide may be fused to another polypeptide to provide for added functionality, e.g. to increase the in vivo stability. Generally such fusion partners are a stable plasma protein, which may, for example, extend the in vivo plasma half-life of the polypeptide when present as a fusion, in particular wherein such a stable plasma protein is an immunoglobulin constant domain. In most cases where the stable plasma protein is normally found in a multimeric form, e.g., immunoglobulins or lipoproteins, in which the same or different polypeptide chains are normally disulfide and/or noncovalently bound to form an assembled multichain polypeptide, the fusions herein containing the polypeptide also will be produced and employed as a multimer having substantially the same structure as the stable plasma protein precursor. These multimers will be homogeneous with respect to the polypeptide agent they comprise, or they may contain more than one polypeptide agent.

The candidate polypeptide agent may be produced by eukaryotic orprokaryotic cells. It may be further processed by unfolding, e.g. heat denaturation, DTT reduction, etc. and may be further refolded using methods known in the art. Modifications of interest that do not alter primary sequence include chemical derivatization of polypeptides, e.g., acylation, acetylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine. The polypeptides may have been modified using ordinary molecular biological techniques and synthetic chemistry so as to improve their resistance to proteolytic degradation or to optimize solubility properties or to render them more suitable as a therapeutic agent. Analogs of such polypeptides include those containing residues other than naturally occurring L-amino acids, e.g. D-amino acids or non-naturally occurring synthetic amino acids. D-amino acids may be substituted for some or all of the amino acid residues.

The candidate polypeptide agent may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like. Alternatively, the candidate polypeptide agent may be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In some cases, the candidate polypeptide agents to be screened are antibodies. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. The specific or selective fit of a given structure and its specific epitope is sometimes referred to as a “lock and key” fit. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammal, chicken, other avians, etc., are considered to be “antibodies.” Antibodies utilized in the present invention may be either polyclonal antibodies or monoclonal antibodies. Antibodies are typically provided in the media in which the cells are cultured.

Agents may be obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Candidate agents are screened for biological activity by adding the agent to at least one and usually a plurality of cell samples, usually in conjunction with cells lacking the agent. The change in parameters in response to the agent is measured, and the result evaluated by comparison to reference cultures, e.g. in the presence and absence of the agent, obtained with other agents, etc.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the selected parameters. For example, luminometry may be employed to detect luciferase or β-galactosidase activity. Flow cytometry may be employed to detect luciferase activity, β-galactosidase activity, or fluorescence activity from fluorescent proteins. Western blots may be employed to assay proteins intracellularly and/or secreted into the medium. Such methods would be well known to one of ordinary skill in the art.

Other uses of the compositions and methods disclosed herein include the generation of in vivo data for modeling aggregation processes in the cellular environment; the validation of in vitro data, e.g. the effect of point mutations on the aggregation of amyloidogenic proteins; the proteomic analysis of interacting partners so as to identify new therapeutic targets; and the analysis of changes in gene expression that are induced by intracellular versus extracellular aggregation, as appreciated by the ordinarily skilled artisan and as described further below.

### Reagents and Kits

Also provided are reagents and kits thereof for the preparation and/or use of aggregation sensors. The subject reagents and kits thereof may vary greatly, and may include one or more of the following: nucleic acids encoding aggregation sensors, e.g. as vectors or as linear DNA for insertion into a vector of choice; host cells, e.g. cells into which a nucleic acid encoding an aggregation sensor may be introduced, or cells stably expressing an aggregation sensor; reagents for inducing the expression of the aggregation sensor, if the sensor is under the control of an inducible promoter; positive and negative control vectors or host cells comprising integrated positive and/or negative control sequences, etc. The various reagent components of the kits may be present in separate containers, or some or all of them may be pre-combined into a reagent mixture in a single container, as desired.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another form of these instructions is a computer readable medium, e.g., diskette, compact disk (CD), etc., on which the information has been recorded. Yet another form of these instructions that may be present is a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

Formation of intra- and extracellular protein aggregates is tightly linked to many neurodegenerative diseases including Alzheimer's disease (AD) (J. A. Hardy, G. A. Higgins, 1992, Alzheimer's disease: the amyloid cascade hypothesis. Science 256, 184; A. Aguzzi, C. Haass, 2003, Games played by rogue proteins in prion disorders and Alzheimer's disease. Science 302, 814; D. J. Selkoe, 2004, Cell biology of protein misfolding: the examples of Alzheimer's and Parkinson's diseases. Nat Cell Biol 6, 1054). AD is the most prevalent neurodegenerative disorder and is estimated to account for 60-80% of the 35 million cases of dementia recorded worldwide in 2010. Genetic, neuropathological and biochemical studies point to Aβ aggregation as a critical step in the pathogenesis of AD (Aguzzi, C. Haass, 2003, supra; B. Caughey, P. T. Lansbury, 2003, Protofibrils, pores, fibrils, and neurodegeneration: separating the responsible protein aggregates from the innocent bystanders. Annu Rev Neurosci 26, 267; S. Lesne et al., 2006, A specific amyloid-beta protein assembly in the brain impairs memory. Nature 440, 352). Sequential proteolysis of the type I membrane glycoprotein amyloid precursor protein (APP) by β-secretase and the γ-secretase complex results in formation of the 38 to 43 amino-acid peptide Aβ (D. J. Selkoe, 2004, supra; C. Haass, B. De Strooper, 1999, The presenilins in Alzheimer's disease—proteolysis holds the key. Science 286, 916). Aβ40 and the faster aggregating Aβ42 are the major forms of Aβ found in AD brains (D. J. Selkoe, 2004, supra; B. Caughey, P. T. Lansbury, 2003, supra). Processing and subsequent Aβ aggregation occur as APP is trafficked through the secretory and recycling pathways (D. G. Cook et al., 1997, Alzheimer's A beta(1-42) is generated in the endoplasmic reticulum/intermediate compartment of NT2N cells. Nat Med 3, 1021; M. Khvotchev, T. C. Sudhof, 2004, Proteolytic processing of amyloid-beta precursor protein by secretases does not require cell surface transport. J Biol Chem 279, 47101). In addition, there is evidence that Aβ can aggregate intracellularly either because a portion of Aβ is not secreted or because secreted Aβ is taken back up by the cell (D. M. Walsh et al. 2000, The oligomerization of amyloid beta-protein begins intracellularly in cells derived from human brain. Biochemistry 39, 10831).

Modulation of Aβ aggregation may have therapeutic benefits for the prevention and/or treatment of AD, but despite wide efforts, the identification of modulators of in vivo Aβ aggregation has posed a significant challenge. While some small molecule aggregation inhibitors have been reported, no clinically useful, disease modifying, therapeutics have emerged. Large-scale screens for chemical or genetic modulators of in vivo Aβ aggregation have been hampered by the lack of cost-effective quantitative models, which replicate relevant subcellular compartments in mammalian neurons, but are also amenable to high-throughput screening. Hence, systematic, high-throughput approaches to find small molecule and genetic modulators of in vivo Aβ aggregation require the development of new quantitative assays that reflect these differing cellular complexities.

We addressed this problem by developing genetically encoded, highly sensitive, bioluminescent sensors that enabled us to monitor and quantify protein aggregation in mammalian neurons and intact brains.

### Materials and Methods

The following materials and methods were used throughout the Examples presented herein.

DNA Constructs for Protein Expression

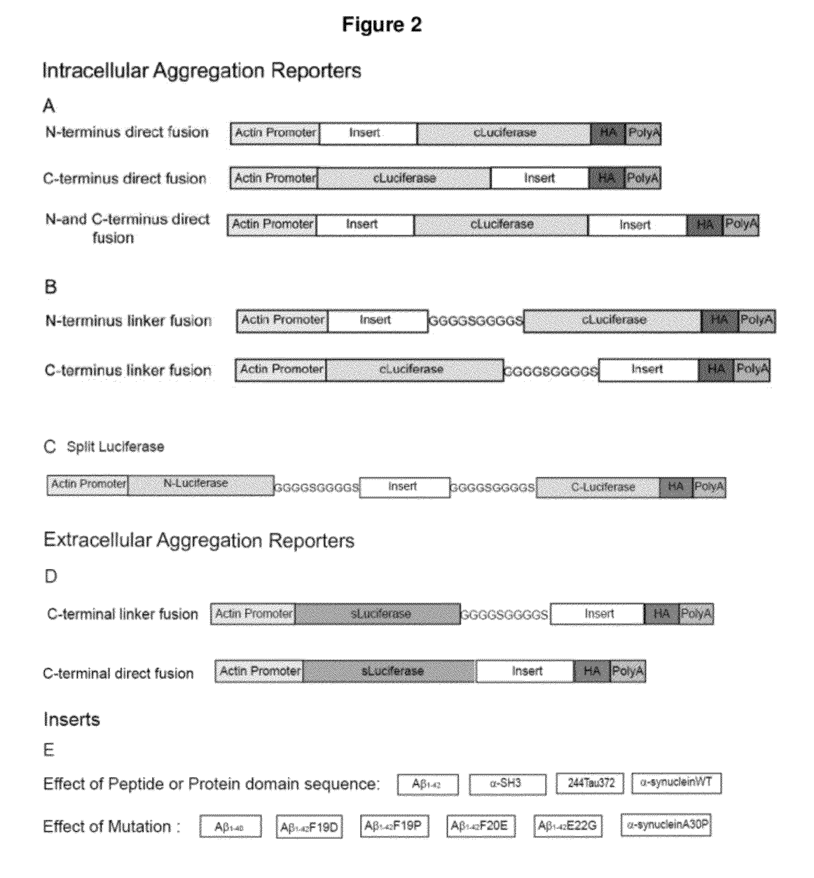

A firefly luciferase (cLuc) expression vector was purchased from Promega, and Metridia Longa luciferase (sLuc) expression vector from Clontech. DNA fragments with XhoI/SalI sites were generated by PCR using Phusion High-Fidelity PCR Master kit from Finnzymes (Espoo, Finland), and cloned into an mammalian expression vector with a CMV-IE enhancer and Beta actin promoter (FIG. 2). The α-spectrin SH3 cDNA was synthesized by GenScript USA Inc. The Aβ42 cDNA was a kind gift of Dr. Bingwei Lu. Inserts were generated by high-fidelity PCR with XhoI/SalI sites. Point mutations of Aβ42 were introduced using the QuikChange II Site-Directed Mutagenesis Kit (Stratagene). For the generation of isogenic inducible 293T cell lines, C-terminal linker Aβ42 and SH3 fusion constructs to cLuc and sLuc, were subcloned into the pCIG-vector containing the CAGGS promoter, and IRES-(NLS)3EGFP (S. G. Megason, A. P. McMahon, 2002, Development 129, 2087). The reporter, cLuc or sLuc, C-terminal linker fusion to Aβ42 or SH3-IRES-(NLS)3EGFP insert was generated by high-fidelity PCR, and cloned into the pcDNA5/FRT/TO (Invitrogen). The gene of interested is expressed from the CMV promoter and regulated by the Tet repressor.

Cell Culture and Transfection

HEK293T cell lines (293T). 293T cells were grown in DMEM (Gibco) supplemented with 10% Heat Inactivated Fetal Bovine Serum (ATCC), 2 mM L-Glutamine (Gibco), 2% HEPES (Gibco), and Penicillin/Streptomycin (Gibco). For transient transfections, cells were plated in 96- or 24-wells plates in medium without antibiotics, and transfected with Lipofectamine 2000 following manufacturer's recommendations (Invitrogen). 6-7 hours post-transfection cells were switched into medium containing antibiotics. Clasto-betalactacystin (Calbiochem) was reconstituted in molecular biology grade Dimethyl sulfoxide (DMSO, Sigma-Aldrich). Cell medium was prepared containing 2× of the final lactacystin concentration, 10 μM for intracellular reporter and 5 μM for secreted reporters, because 10 μM was found to be toxic in transfections with the secreted reporters, and diluted to 1× with the medium in the well. Control cells were treated with medium containing the same volume of solvent. Cells were harvested after 40 hours: Cell culture supernatant was collected for secreted luciferase, and cells were lysed with Luciferase Passive Lysis Buffer (Promega) or RIPA buffer containing protease inhibitors.

293T FlipInTrex (Invitrogen) cells. 293T FlipInTrex cells were grown as described above, using Tet Approved Fetal Bovine Serum (Clontech) and supplementing the medium with Zeocin 100 μg/mL (Invitrogen) and Blasticidin 15 μg/ml (InVivoGen, San Diego, USA). Zeocin selects for the lacZeo inserted into FRT sites in the genome, and Blasticidin selects for Tet repressor expression. For the generation of isogenic stable doxycycline inducible cell lines, cells were co-transfected overnight using Lipofectamine 2000 with the pcDNA5/FRT/TO vectors containing the fusion proteins-IRES-(NLS)₃EGFP cassettes, and the pOGG-Flp recombinase plasmid (Invitrogen) at a ratio pcDNA/pOGG of 1:9. On day 2, cells were replated at −25% confluency, and selection for recombined clones was started by supplementing the medium with 75 μg/ml Hygromycin (InvivoGen) and Zeocin 100 μg/mL. Medium was exchanged every 2-3 days until colonies appeared. Cells were adapted to cell culture media containing 5% Tet approved Fetal Bovine Serum (Clontech), and 5% Serum Replacement 3 (Sigma). Hygromycin and Zeocin selection were kept through passages to ensure that the cell lines do not lose the inserted DNA, or Tet repressor expression. Expression of the chimeric reporter proteins was induced by addition of 0.75 μg/mL Doxycycline (Sigma-Aldrich) to cell culture medium, and tested for luciferase activity and western-blot (see below).

Kinetic Experiments. Cells were plated in 96-well plates, and allowed to attach overnight. Cell medium without antibiotics was prepared containing 2× of the final doxycycline concentration, 0.75 μg/ml, and diluted to 1× with media in the well. Fresh doxycycline was added to the medium every 24 hrs to keep protein induction constant. 8 wells were treated per time point. At different times of induction, 4 wells were harvested for luciferase assays, and 4 wells were used for cell survival assays (see below for luciferase and cell survival assays).

Effect of Small Molecule Inhibitors of Aβ42 aggregation in inducible cell lines. Congo Red, Rosmarinic acid, Quercetin, scyllo-inositol, and (−)-Epigallocatechin gallate (EGCG) were purchased from Sigma-Aldrich, and dissolved in DMSO. Cells were plated in 96-well plates and allowed to attach overnight. Cell medium without antibiotics was prepared containing 2× of the final compound concentration (1 μM) and 2× of final doxycycline concentration (0.75 μgr/ml) and diluted to 1× with media in the well. Control cells were treated with medium containing the same volume of DMSO. 8 wells were treated per condition. After 24 hours, 4 wells were harvested for luciferase assays, and 4 wells were used for cell survival assays.

Primary Hippocampal cultures. The hippocampi of PO mice were dissected, pooled, dissociated and transfected with the Mouse Neuron Nucleofection Kit (Lonza AG) using an Amaxa Nucleofector II. Cells were cultured as previously described (I. A. Graef et al., 1999, Nature 401, 703) and harvested for luciferase assays after 48 hours.

Enzymatic Activity and Cell Survival Assays.

cLuciferase Firefly activity. Firefly luciferase reagent was prepared as described in (B. W. Dyer, et al., 2000, Anal Biochem 282, 158). Reagents (D-Luciferin, Acetyl Coenzyme-A, EGTA, ATP, DTT, Gly-Gly buffer, MgSO4) were purchased from Sigma-Aldrich. 20 μl of cellular lysate were transferred to white 96-well plates (Corning), 100 μl of luciferase reagent were injected, and every well was read for 5 s followed by 3 s delay to minimize cross-talk between wells using a Modulus Microplate Multimode Reader (Turner Biosystems Inc., Sunnyvale, Calif., USA). cLuciferase activity is measured in Relative Luminiscence Units (RLU).

sLuc Metridia activity. sLuc Metridia activity was measured using the same plates and instrument described above. Metridia reagent was prepared as described in (G. A. Stepanyuk et al., 2008, Protein Expr Purif 61, 142). Coelenterazine, native (Biosynth AG, Staad, Switzerland) was brought up at 1.43 mM in Methanol acidified with 1N HCl and diluted to 7.2 μM in 20 mM Tris-HCl 0.3M NaCl pH7.5 (Sigma-Aldrich). 20 μl of cell medium were transferred to white 96-well plates, 50 μl of reagent was injected, and every well was read for 5 s followed by a 3s delay. sLuciferase activity is measured in Relative Luminiscence Units (RLU).

β-Galactosidase activity. β-Galactosidase activity was measured using an adaption of the method described in (J. G. Sambrook, Russell, D. W., 2006, Cold Spring Harb Protoc. doi:10.1101/pdb.prot3952). o-nitrophenyl-β-D-galactopyranoside (ONPG, Sigma-Aldrich) was reconstituted in 0.1 M phosphate buffer at 20 mg/ml, and diluted to 1 mg/ml in assay buffer containing β-Mercaptoethanol (Sigma-Aldrich). 100 μl of ONPG reagent were added to 5 or 10 μl of lysate in clear polystyrene 96-well plates (Fisher Scientific). Plates were shaken for 30 s and incubated in the dark at 37° C. for 1 hour. 150 μl of Tris 1M pH8 were added to stop the reaction, and the absorbance at 420 nm was recorded in a Spectramax M5 plate reader (Molecular Devices, Sunnyvale, Calif., USA).

Cell survival was measured using Cell titer Blue kit (Promega). Cells were plated in 96-well plates, and after the incubation time, 10 μl of cell titer blue reagent were added per well. After 1 hour incubation at 37° C., fluorescence was recorded in a Spectramax M5 plate reader, using excitation at 560 nm and emission at 590 nm in Relative Fluorescence Units (RFU).

In Vitro Aggregation of Synthetic Aβ Peptides

Aβ42, Aβ40, Aβ42F20E and Aβ42E22G peptides (Bachem, Torrance, USA) were disaggregated using Hexafluoro-2-propanol (HFIP, Sigma-Aldrich) following the protocol described elsewhere (C. Goldsbury, et al., 2005, J Mol Biol 352, 282), and aliquoted into low protein binding tubes (Eppendorf).

Effect of small molecule inhibitors of Aβ42 aggregation. 2 mM Aβ42 stock solutions in DMSO) were sonicated for 5 minutes, and diluted to final 25 μM concentration in PBS (Gibco). Small molecules were added to final 1 μM concentration, the final DMSO concentration in all samples and control was kept equal. Samples were incubated for 1 day at 30° C., and tested for Aβ42 aggregation using Thioflaving T (Tht, Sigma-Aldrich) binding monitored by fluorescence (excitation 450 nm/emission 485 nm). Tht was prepared at 5 μM in 20 mM Glycine pH=8 buffer. 10 μl of sample were plated in triplicates in clear bottom non-binding black plates (Corning), and 100 μl of Tht were added. After 5 minutes of incubation at room temperatures, fluorescence was read. Data were corrected by Tht alone fluorescence, and normalized by the Tht intensity of the control solution, Aβ42+DMSO. Ratios and standard deviations were calculated as described in the Data Analysis section.

In vitro Aβ42 kinetic experiment. 25 μM Aβ42 in PBS was incubated at 30° C. Aliquots were withdrawn at different time points, and Tht binding was measured in triplicates.

In vitro aggregation of Aβ42 mutants. Aβ40 and Aβ42 were incubated as described above. Aβ42 F20E and Aβ42 E22G were incubated as previously described (L. M. Luheshi et al., 2007, PLoS Biol 5, e290; A. S. Johansson et al., 2006, FEBS J 273, 2618) using matching conditions for Aβ42. All peptides were incubated for two days, and tested for Tht binding. Inhibition ratios were calculated as the Tht binding intensity of Aβ-mutant/Aβ42-wt under the same experimental conditions. Ratios and standard deviations were calculated as described in the Data Analysis section.

Electron Microscopy of Aβ42 aggregation kinetics was performed as previously described (M. Lopez De La Paz et al., 2002, Proc Natl Acad Sci USA 99, 16052) and imaged in a JEOL TEM1230 microscope.

Immunoblotting and Immunofluorescence

Primary antibodies: Goat Anti-Firefly Luciferase-HRP (cLuc, Abcam); Mouse anti HA-tag, rabbit anti-mTor, rabbit anti-Hsp70, rabbit anti-Bip (Cell signaling); Mouse Anti-Aβ1-16 (6E10, Covance); Monoclonal Anti-β-Actin-HRP and rabbit anti-Ulk2 (Sigma-Aldrich).

Secondary antibodies: Anti-mouse-HRP and anti-rabbit HRP (Jackson's Laboratories), anti-rabbit HRP (Cell signaling), Anti Mouse Alexa Fluor-594 (Invitrogen).

Immunoblotting: For extracellular protein detection, protease inhibitors were added to collected cell media, and samples were centrifuged to remove cellular debris, and quantified using a Bradford assay. Typically 75 μg of total protein were run under denaturing conditions in 12% SDSPAGE gels. Dura ECL reagent (Pierce) was used to detect HRP activity. Intracellular lysates were prepared in RIPA buffer containing protease inhibitors and cleared by ultracentrifugation. Protein concentration was determined using a Bradford test (Thermo Scientific) and BSA (Pierce) for the standard curve. 30-40 μg of total protein lysates were run under denaturing conditions in 10% SDS-PAGE gels, and transferred onto nitrocellulose membranes. Immunoblotting was carried out following standard procedures, followed by HRP activity detection using ECL reagent (Pierce).

Immunofluorescence: Cells were grown on glass cover slips coated with poly-L-ornithine (Sigma Aldrich) and Fibronectin (Invitrogen), and fixed in 4% Paraformaldehyde (Electron Microcopy Sciences) for 30 min at room temperature. Following rinses with PBS, cells were incubated in blocking buffer (5% goat serum/0.01% TritonX-100) for 1 hour at room temperature. Primary antibody incubations were carried out at 4° C. in blocking buffer, washed 3 times, and incubated in blocking buffer containing secondary antibody and DAPI (Sigma-Aldrich) for 1 hour at room temperature, washed in PBS 3 times and mounted onto glass slides. Imaging was done with a Leica DM5000B Fluorescence microscope using a 63× HCS PL APO oil immersion objective lens, and a Leica DM 6000 B confocal microscope, using a 100× HCX PL APO oil immersion objective. For aggregate counting, 7 independent fields were photographed per sample at 63× magnification in the fluorescence microscope. GFP positive cells and aggregate positive cells were manually counted. The plots in the paper show the average of two independent countings. Statistical significance was calculated using a two tailed Student's t-test in Prism software.

Filter Trap Assays

Filter trap assays were performed using a modification of previously published protocols (E. E. Wanker et al., 1999, Methods Enzymol 309, 375) Cell culture media containing the sLuc reporters was harvested at different times of induction, 1 mM PMSF was added and frozen at −80° C. for storage. Aliquots were centrifuged briefly to remove cellular debris, and protein concentration was measured using a Bradford test. Cellulose acetate membrane with 0.2 μm pore size (Whatman) was pre-rinsed in 1% SDS PBS. Cell medium containing 200 μg of total protein and 1% SDS were filtered through cellulose acetate membranes using a Bio-dot Microfiltration apparatus (Bio-Rad), and washed once with PBS/1% SDS. Proteins were detected as described for Western-Blots. Integrated density was calculated using ImageJ (NIH) and stastistical significance was calculated as above.

Quantitative Real-Time PCR (qRT-PCR)

Total RNA from stably transfected 293T FlipInTrex induced for 96 hrs was purified using Trizol treatment (Invitrogen) followed by DNase digestion (Qiagen) and RNA isolation using RNeasy MinElute Cleanup Kit (Qiagen). cDNA was synthesized using random hexamers and Superscript III reverse transcriptase (Invitrogen) according to manufacturer's instructions. Primers were designed using Primer3 (S. Rozen, H. Skaletsky, 2000, Methods Mol Biol 132, 365) and experimentally tested for replication efficiency. qRT-PCR analysis was performed using GAPDH (Glyceraldehyde 3-phosphate dehydrogenase) as internal control for normalization. Each reaction contained 1 μL cDNA template, 100 or 200 pmol of each primer, and 1×SYBR green supermix (Applied Biosystems) to a final volume of 20 μL. Reactions were carried out using a StepOne Plus Real-Time PCR System (Applied Biosystems) for 40 cycles (95° C. for 15 s, 60° C. for 60 s). The purity of the PCR products was determined by melt curve analysis. Relative gene expression was calculated using the change in cycling threshold method (ΔΔCt) (T. D. Schmittgen, K. J. Livak, 2008, Nat Protoc 3, 1101) with DataAssist v2.0 Software (Applied Biosystems). Expression levels of triplicate PCR samples were normalized to the levels of GAPDH. Data is reported as the ratio of the normalized mean expression levels of the Aβ-tagged reporter cell line versus the SH3-tagged reporter cell line.

In Utero Electoporation and Bioluminiscence Brain Imaging.

pCIG-plasmids (see cloning section) containing cLuc-SH3 or cLuc-Aβ-IRES-(NLS)3EGFP were transfected into forebrain of E14.5CD1 embryos by in utero electroporation as described (T. Saito, 2006, Nat Protoc 1, 1552). Briefly, the expression plasmid was injected into embryonic forebrain with concentration of 2 μg/μl in a total volume of 2 μl. Embryonic brains were electroporated with five 40V electronic pulses at 1s intervals using a BTX electroporator (Electro Square Porator ECM830). Embryos were harvested at E17.5. Transfected embryos were identified by GFP fluorescence and images were taken using a Leica MZ16FA Motorized Fluorescence Stereo microscope. Whole GFP+ brains (cLuc-Aβ n=3, cLuc-SH3 n=2) were imaged for cLuc activity by submerging them into PBS containing 15 μgr/ml D-Luciferin (Biosynth Inc.), using an IVIS Spectrum imaging system at the Stanford Center for Innovation in In-Vivo Imaging (SCI3). Data were analyzed using Living Imaging 3.2 Software (Xenogen, Caliper Life Sciences). A region of interest (ROI) was manually selected. Only expression in cortical areas was considered for the analysis. The area of the ROI was kept constant and the intensity was recorded as total flux of photons [photons_s−1] within a ROI. Area of GFP expression was quantified using Image J (NIH). EGFP-expressing areas and control areas were dissected to prepare protein lysates using RIPA buffer containing protease inhibitors (cLuc-Aβ n=2, cLuc-SH3 n=3), or to prepare luciferase extracts (cLuc-Aβ n=2, cLuc-SH3 n=3) as reported (M. Manthorpe et al., 1993, Hum Gene Ther 4, 419).

Data Normalization and Statistics.

Expression of chimeric luciferase constructs in HEK293T cells. Luciferase, β-Galactosidase and cell survival readings were performed in quadruplicates. Data normalization was carried out by dividing the average of a luciferase reading by the average of the β-Galactosidase or cell survival reading. The standard deviation of the ratio was calculated using error propagation theory. An example is shown below.

Let L={I1, I2, I3, I4} represent the luciferase measurement technical replicates, and B={b1, b2, b3, b4} represent the β-Gal technical replicates. If β exists and b1 . . . b4>0, the stochastic variable Z represents the ratio of the averages then

$$Z = \frac{\overline{L}}{\overline{B}} , \{ {b}_{1 } … {b}_{4} > 0 \} \\ var ( z ) = {( \frac{\overline{L}}{\overline{B}} )}^{2} ( \frac{var ( L )}{{\overline{( L )}}^{2}} + \frac{var ( B )}{\overline{{( B )}^{2}}} ) \qquad Equation 1$$

and therefore

$$std ( z ) = ( \frac{\overline{L}}{\overline{B}} ) \sqrt{( \frac{{sd ( L )}^{2}}{{\overline{( L )}}^{2}} + \frac{{sd ( B )}^{2} )}{\overline{{( B )}^{2}}} )} \qquad Equation 2$$

Experiments were repeated at least three times to verify trends. Plots in paper show data from a representative experiment.

Expression of chimeric luciferase constructs in primary hippocampal neurons. Luciferase readings were performed in quadruplicates. Data normalization was carried out by dividing the average of the luciferase reading for a given Aβ variants by the average of the luciferase reading for Aβ42 WT. The standard deviation of the ratio was calculated using error propagation theory.

Effect of small molecule inhibitors of Aβ42 in the inducible cell lines. The ratio luciferase activity/cell survival (Rsmall molecule) was calculated as shown in eq. 3. Four technical replicates were measured per molecule for each test. Standard deviation was calculated using eq. 2.

$$Rsmall molecule = \frac{Average luciferase activity}{Average cell survival} \qquad Equation 3$$

Data for a specific molecule in a given experiment was normalized by the ratio for the DMSO treated cells. This ratio is called Fold inhibition.

$$Fold inhibition = \frac{Rsmall molecule}{R DMSO} \qquad Equation 4$$

Fold inhibition ratio >1, indicates that a molecule is able to interfere with Aβ42 induced luciferase aggregation, and therefore, it increases its enzymatic activity compared with the DMSO treated cells. Comparing the Fold inhibition ratio of a given molecule in the Aβ42 and SH3 sensors allows the identification of molecules that change the ratios by mechanisms non-specific to aggregation such as toxicity or interference with luciferase activity. Data shown in the plots are the average of at least 4 independent repetitions per small molecule in every sensor. Standard errors were calculated using Error propagation theory. Statistical significance of the difference in the Fold inhibition ratios of a given molecule in the Aβ42 vs. SH3 sensors was calculated with Prism Software using two-tails unpaired t-tests.

### Example 1

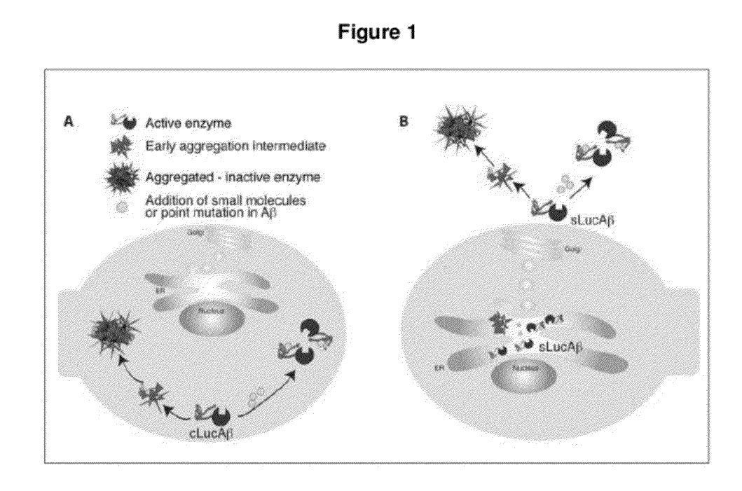

As the question where pathogenic Aβ aggregation occurs and where a genetic or chemical modulator should exert its effect is still unanswered, we developed sensors that model Aβ aggregation in either the cytosolic or the secretory compartment (FIG. 1). The design of the aggregation sensors was based on the well-established principle that fusion of an aggregating peptide, such as Aβ to another protein can trigger misfolding and aggregation of the chimeric protein (W. C. Wigley, et al., 2001, Protein solubility and folding monitored in vivo by structural complementation of a genetic marker protein. Nature Biotechnol. 19, 131). We constructed intracellular and secreted aggregation sensors using the cytoplasmic firefly (cLuc) and secreted Metridia longa (sLuc) luciferase enzymes (FIGS. 1 and 2).

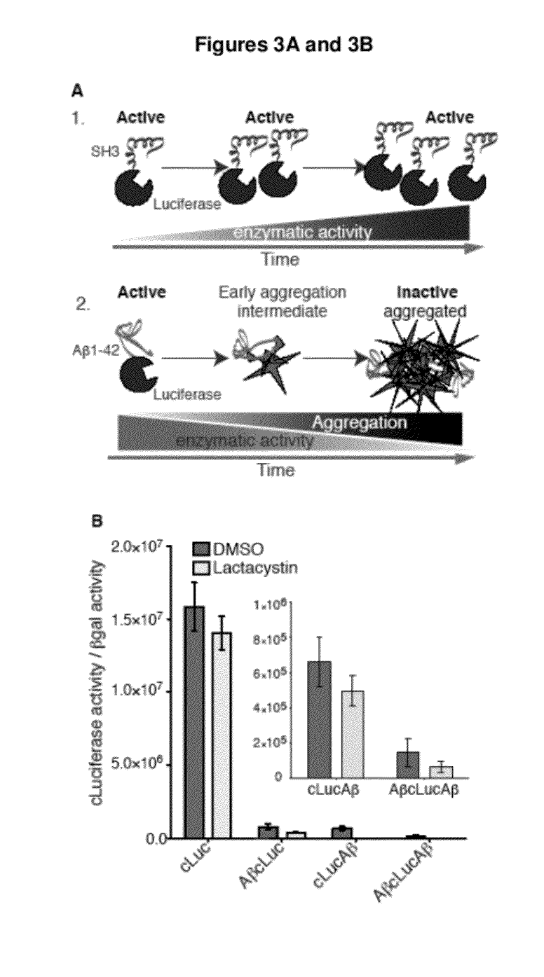

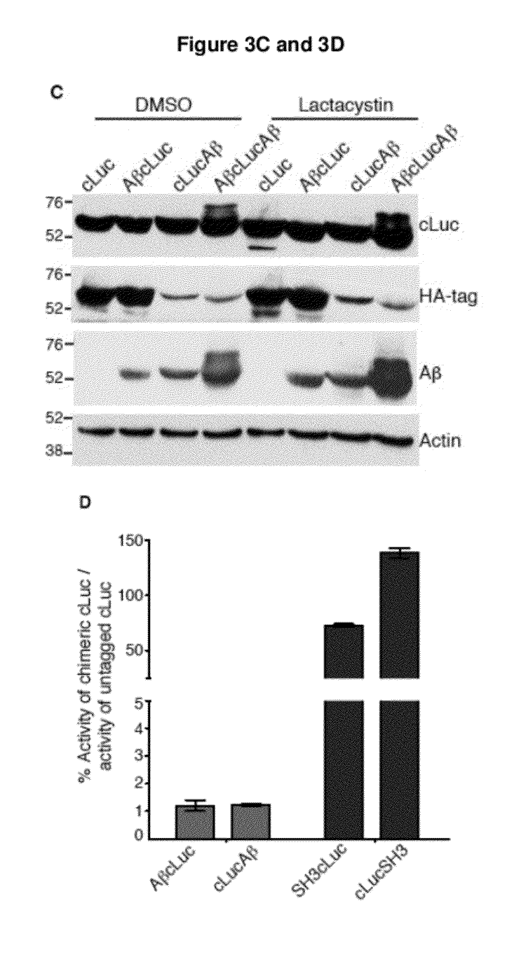

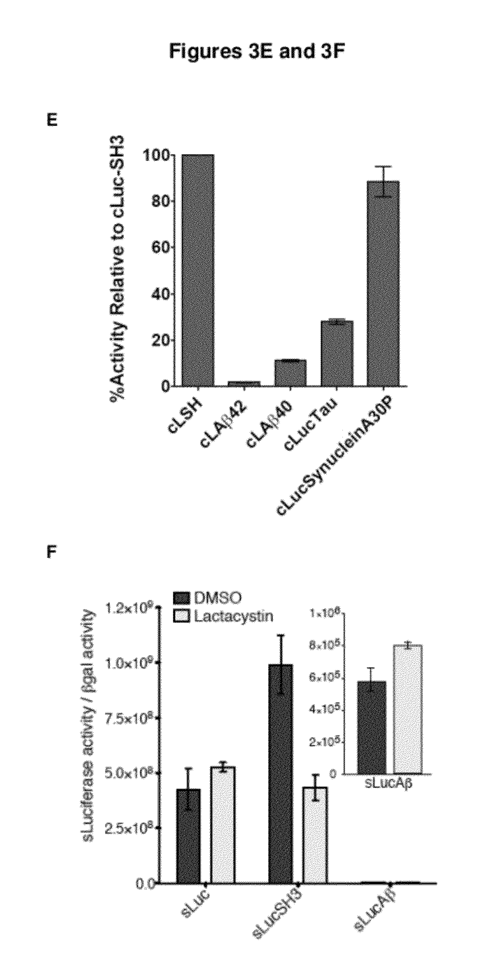

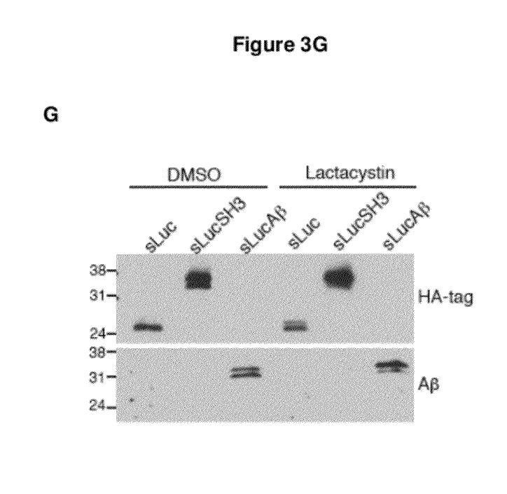

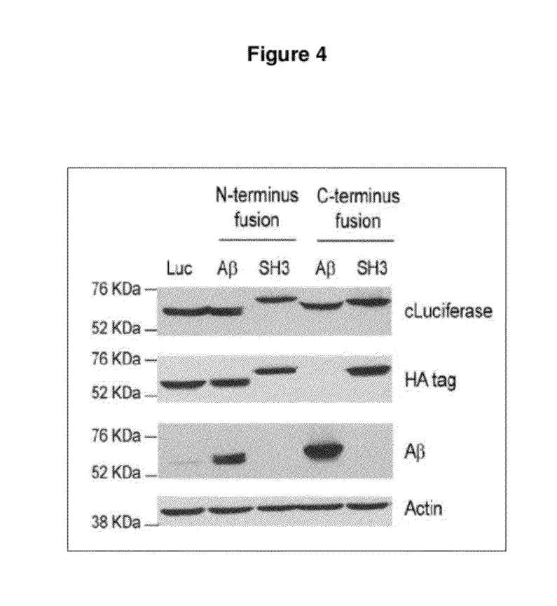

N- or C-terminal fusion of Aβ42 to cLuc resulted in a −40 fold reduction of reporter activity, which was further diminished (˜140 fold) by attachment of Aβ42 to both termini of cLuc (FIGS. 3A and 3B, and Table 1 below). Levels of fusion-protein expression, measured by immunoblotting with an antibody specific for cLuc, were similar (FIGS. 3C and 4). Interestingly, fusion of Aβ42 close to the C-terminal HA-epitope tag resulted in reduced detection with anti-HA antibody (FIGS. 3C and 4). N- or C-terminal fusion of the α-spectrin SH3 domain (a non-aggregating protein of similar size to Aβ; A. Esteras-Chopo et al., 2005, The amyloid stretch hypothesis: recruiting proteins toward the dark side. Proc Natl Acad Sci USA 102, 16672) to cLuc resulted in no reduction of enzymatic activity and had no effect on protein expression (FIGS. 2A, 3D, 4).

TABLE 1
Normalized activity of intracellular cLuc fusion
proteins transiently expressed in 293T cells.

| | DMSO^{(a)} | 10 μM Lactacystin^{(a)} |
|---|---|---|
| cLuc^{(b)} | 1.6 10⁺⁷ ± 1.6 10⁺⁶ | 1.4 10⁺⁷ ± 1.1 10⁺⁶ |
| cAβ₄₂-Luc | 7.5 10⁺⁵ ± 1.9 10⁺⁵ | 3.9 10⁺⁵ ± 2.5 10⁺⁵ |
| cLuc-Aβ₄₂ | 6.6 10⁺⁵ ± 1.4 10⁺⁵ | 4.9 10⁺⁵ ± 8.7 10⁺⁵ |
| cAβ₄₂-Luc-Aβ₄₂ | 1.4 10⁺⁵ ± 8.2 10⁺⁴ | 6.3 10⁺⁵ ± 3.0 10⁺⁵ |

^{(a)}Columns show cluciferase activity (RLU) normalized by cotransfected β-Galactosidase activity (arbitrary units).
^{(b)}All constructs were transiently expressed for 40 hours in the presence of vehicle (DMSO) or 10 μM lactacystin.

We constructed a similar sensor to probe Aβ aggregation in the secretory compartment by fusing sLuc to either Aβ42 (sLucAβ) or the SH3 domain (sLucSH3) (FIG. 2C) and measured expression and activity of secreted reporters in the cell culture supernatant. We found that the activity of sLucAβ was ˜1,700 fold lower than sLuc, while the activity of sLucSH3 was 2 fold higher than sLuc (FIG. 3G and Table 2, below). We observed similar levels of sLuc and sLucAβ protein expression and increased expression of sLucSH3 (FIG. 3G).

TABLE 2
Normalized activity of secreted sLuc fusion
proteins transiently expressed in 293T cells.

| | DMSO^{(a)} | 5 μM Lactacystin^{(a)} |
|---|---|---|
| sLuc^{(b)} | 4.3 10⁺⁸ ± 9.3 10⁺⁷ | 5.3 10⁺⁸ ± 2.1 10⁺⁷ |
| sLucAβ | 5.8 10⁺⁵ ± 1.6 10⁺⁵ | 8.0 10⁺⁵ ± 2.9 10⁺⁵ |
| sLucSH3 | 9.9 10⁺⁸ ± 1.3 10⁺⁸ | 4.3 10⁺⁸ ± 5.8 10⁺⁷ |

^{(a)}Columns show sLuciferase activity (RLU) normalized by contransfected β-Galactosidase activity (arbitrary units)
^{(b)}All constructs were transiently expressed for 40 hours in the presence of DMSO or 5 μM lactacystin. 5 μM Lactacystin was used since 10 μM was toxic for cells expressing sLuciferase proteins.

The observed decrease in enzymatic activity of the chimeric Aβ42-luciferase reporters could either be the result of protein degradation or aggregation. To differentiate between these two possibilities, we assessed the effect of proteasome inhibition with lactacystin. We found that it had no significant effect on activity and expression levels of the chimeric reporters (FIGS. 3B and 3G). These results indicate that fusion of the amyloidogenic Aβ42 peptide to both enzymes led to aggregation of the chimeric protein and to loss of enzymatic activity.

Finally, we tested whether the cLuc aggregation sensor would reflect the aggregation propensity of other proteins associated with human neurodegenerative diseases. In primary cortical neurons, fusion of Aβ₄₀ to cLuc resulted in 5 fold higher sensor activity of cLucAβ₄₀ compared to cLucAβ₄₂, which is consistent with a reduced aggregation rate of cLucAβ₄₀. Fusion of the four-repeat domain of human Tau (₂₄₄Tau₃₇₂), which contains two hexapeptide motifs that promote PHF aggregation by formation of β-structure (²⁷⁵VQIINK²⁸⁰ in R2 and ³⁰⁶VQIVYK³¹¹) (Khlistunova et al., 2006, J Biol Chem. 281(2):1205-14), to the C-terminus of cLuc reduced the enzymatic activity to 20% of the non-aggregating cLucSH3 reporter (FIG. 3D). Similarly, fusion of a mutant version of α-synuclein found in early onset familial PD comprising a substitution at residue 30 (“A30P”) resulted in a 25% decrease of reporter activity (FIG. 3D). This last result is in good agreement with recent results that show that factors such as mutations, post-translational modifications, or environmental changes associated with aging trigger α-synuclein aggregation (Bartels, Choi et al. 2011; Wang, Perovic et al. 2011).

### Example 2

Fusions of proteins to reporter genes are often spaced by a linker region of serines/or glycines to provide flexibility and polarity (A. Esteras-Chopo et al., 2005, Proc Natl Acad Sci USA 102, 16672). We generated and expressed N- or C-terminal fusions of Aβ42 and SH3 spaced from cLuc by a linker region (FIG. 2 and Table 3). The presence of the linker does not significantly change the loss of activity of the Aβ42 fusions, but it seems to further stabilize the α-SH3 insertions. We decided to use the C-terminal fusion linker region design for the generation of the secreted luciferase fusion constructs, and the effect of Aβ42 mutations for both cLuc and sLuc.

TABLE 3
Percentage of enzymatic activity of chimeric Aβ42
or SH3 N- or C-terminal cLuc proteins with and
without linker relative to cLuc in 293T cells.

| | SH3^{(a)} | Aβ₄₂^{(a)} |
|---|---|---|
| X-cLuc^{(b)} | 72.9 ± 1.3 | 1.2 ± 0.2 |
| X-linker-cLuc | 123.0 ± 4.1 | 1.5 ± 0.2 |
| cLuc-X | 138.4 ± 4.5 | 1.23 ± 0.04 |
| cLuc-linker-X | 131.2 ± 3.9 | 1.73 ± 0.06 |

^{(a)}Columns show the activity of the fusion construct/activity cLuciferase *100. Activity refers to clLuciferase activity (RLU) normalized by cotransfected β-Galactosidase activity (arbitrary units).
^{(b)}All constructs were transiently expressed for 40 hours.

### Example 3

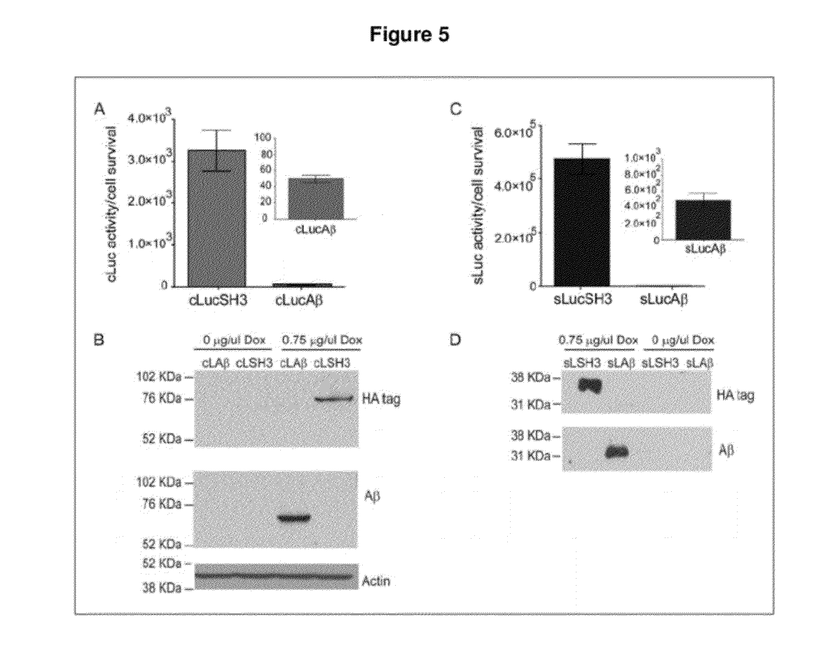

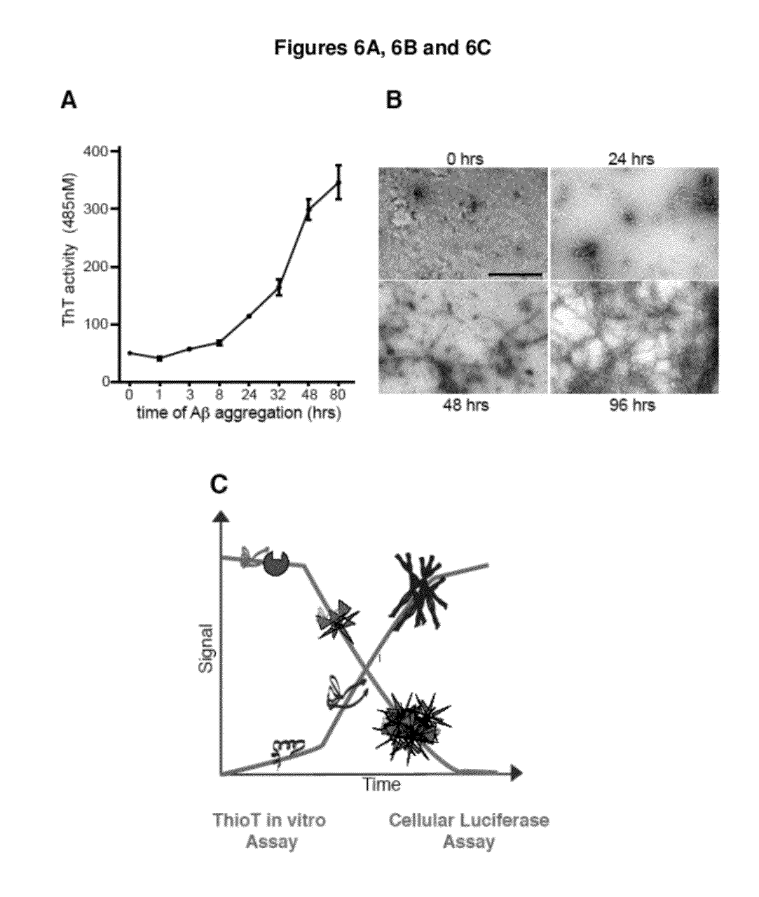

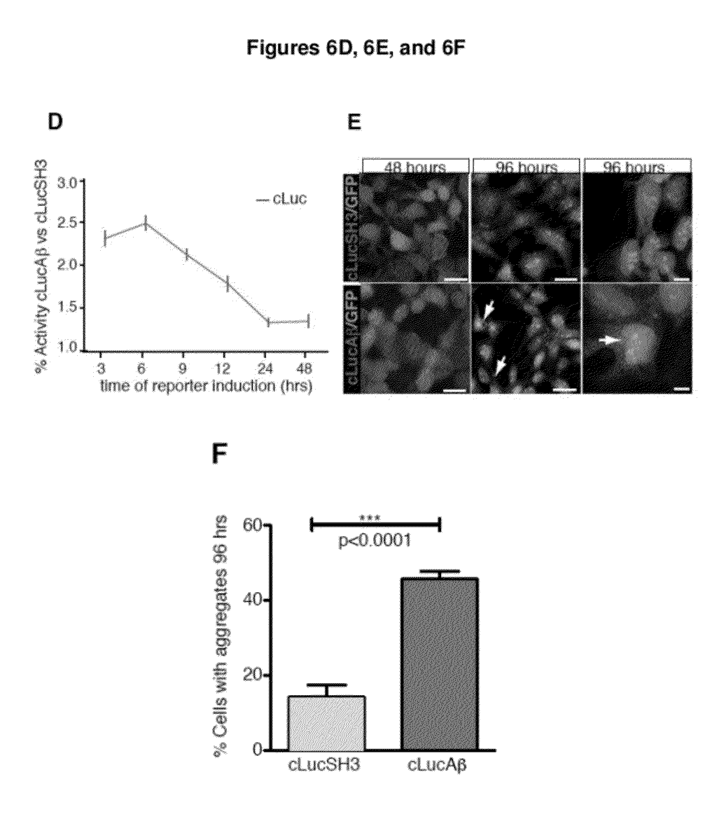

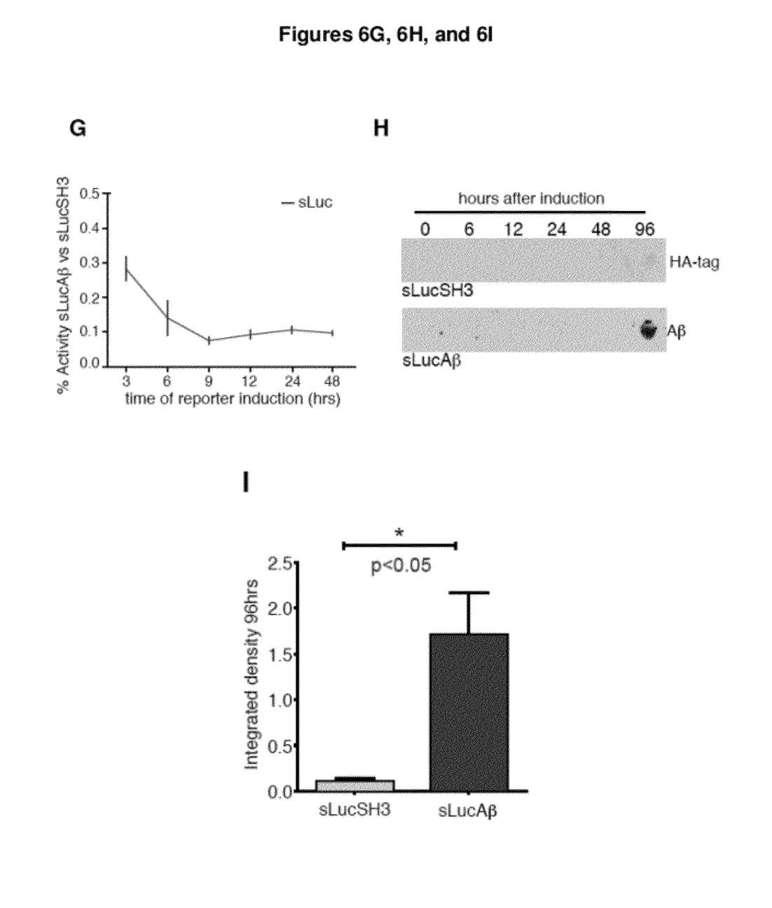

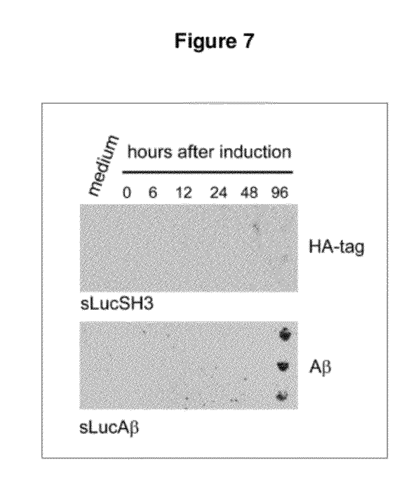

To characterize the aggregation kinetics of the intracellular and secreted bioluminescent sensors, we generated single insert tetracycline (Tet)-inducible 293T cell-lines, which expressed the chimeric reporters (FIG. 5) and used several independent techniques to monitor aggregation. In vitro aggregation kinetics of synthetic Aβ peptide followed a nucleated model (J. T. Jarrett, P. T. Lansbury, Jr., 1993, Seeding “one-dimensional crystallization” of amyloid: a pathogenic mechanism in Alzheimer's disease and scrapie? Cell 73, 1055) (FIGS. 6A and 6C); the initial lag phase (0-6 hours) was followed by an exponential phase (6-24 hours) and reached a plateau (24-72 hours). Different aggregation intermediates grew during this process (FIG. 6B). To test whether the kinetics of reporter activity were consistent with an aggregation model, we compared the relative enzymatic activity of the aggregation sensors (cLucAβ, sLucAβ) to the respective control reporters (cLucSH3, sLucSH3). We found that the cytoplasmic aggregation sensor showed a 6 hour lag phase followed by exponential decay and signal stabilization after 24 hours, which is consistent with the nucleation model (FIG. 6D), while the activity of the sLucAβ rapidly declined within the first 9 hours (FIG. 6G). To determine whether we could detect cellular aggregate formation, we used fluorescence microscopy to monitor the intracellular distribution of chimeric cLuc proteins. At 48 hours of induction, cLucSH3 and cLucAβ were distributed in a homogenous, diffuse pattern (FIG. 6E). In contrast, after 96 hours, cLucAβ was concentrated in large, often juxtanuclear, structures that were reminiscent of inclusion bodies characteristic of intracellular protein aggregation (R. R. Kopito, 2000, Aggresomes, inclusion bodies and protein aggregation. Trends Cell Biol 10, 524), while cLucSH3 was still homogenously distributed (FIGS. 6E and F). To directly examine aggregate formation of the secreted aggregation sensor we performed filter trap assays using the cell culture supernatant (E. Scherzinger et al., 1999, Self-assembly of polyglutamine-containing huntingtin fragments into amyloid-like fibrils: implications for Huntington's disease pathology. Proc Natl Acad Sci USA 96, 4604). We found that while after 96 hours sLucAβ formed large aggregates that were retained by the filter-trap, sLucSH3 remained soluble during the time-course of induction (FIGS. 6H, 6I, and 7).

### Example 4

The data described above indicated that the reduction of enzymatic activity of the Aβ-fusion proteins was most likely the result of protein aggregation. To assess whether the bioluminescent sensors were sensitive enough to detect subtle changes in protein aggregation, we made use of previously identified point mutations that change the aggregation propensity of Aβ. These mutations are found within a hydrophobic stretch in the central part of Aβ42, which is thought to be critical for aggregation and fibrillogenesis (C. Wurth, et al., 2002, Mutations that reduce aggregation of the Alzheimer's Abeta42 peptide: an unbiased search for the sequence determinants of Abeta amyloidogenesis. J Mol Biol 319, 1279). F19P (W. C. Wigley, et al., 2001, Protein solubility and folding monitored in vivo by structural complementation of a genetic marker protein. Nature Biotechnol. 19, 131), F19D (N. S. de Groot et al., 2006, Mutagenesis of the central hydrophobic cluster in Abeta42 Alzheimer's peptide. Side-chain properties correlate with aggregation propensities. FEBS J 273, 658) and F20E (L. M. Luheshi et al., 2007, Systematic in vivo analysis of the intrinsic determinants of amyloid Beta pathogenicity. PLoS Biol 5, e290) had been shown to reduce Aβ aggregation, while the naturally occurring arctic (E22G) mutation, which gives rise to early onset AD, accelerates Aβ aggregation (C. Nilsberth et al., 2001, The ‘Arctic’ APP mutation (E693G) causes Alzheimer's disease by enhanced Abeta protofibril formation. Nat Neurosci 4, 887). We also used Aβ40, which has slower aggregation kinetics than Aβ42 (J. T. Jarrett et al., 1993, The carboxy terminus of the beta amyloid protein is critical for the seeding of amyloid formation: implications for the pathogenesis of Alzheimer's disease. Biochemistry 32, 4693).

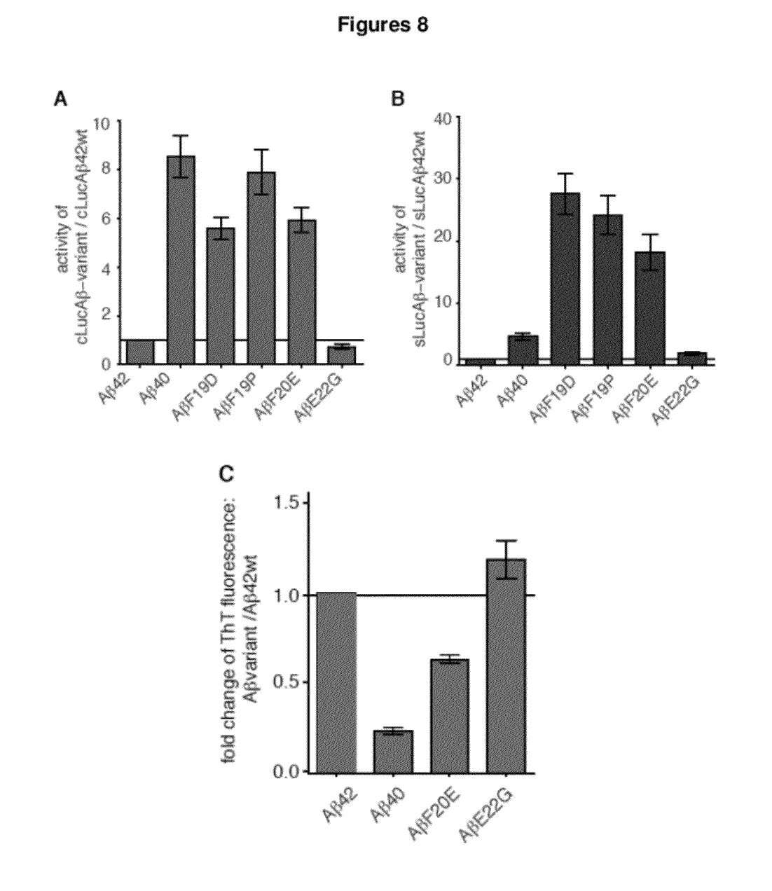

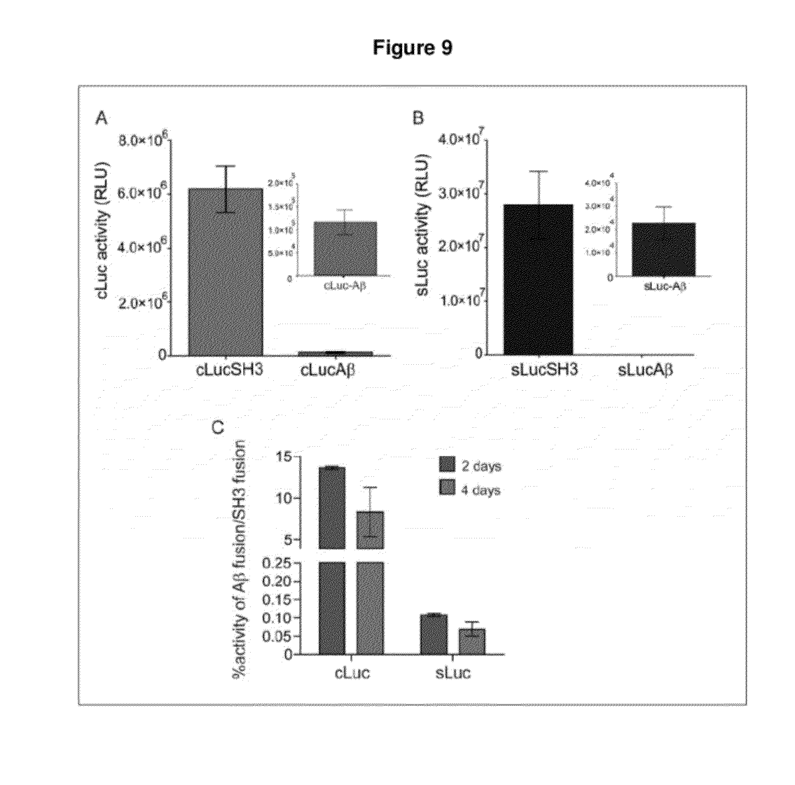

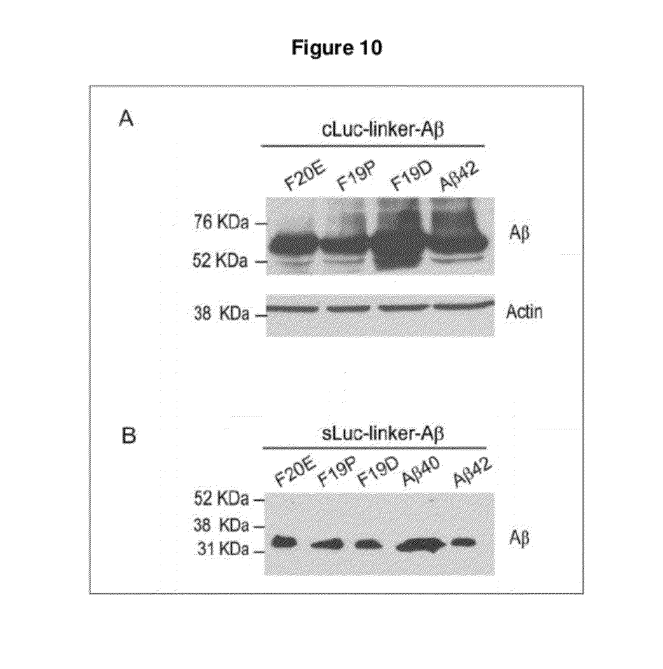

We fused the Aβ variants to the intracellular and secreted sensors and expressed the chimeric reporters in primary hippocampal neurons (FIGS. 8A, 8B, and 9). Consistent with conclusions that the observed reduction in enzymatic activity was the result of Aβ aggregation, variants (Aβvar) which are known to reduce Aβ aggregation increased the activity of the reporters relative to wild-type Aβ42 (ratio Aβvar/Aβ42 wt>1) (FIGS. 8A and B). Interestingly, the extent of this increase seemed to depend not only on the position and nature of the mutation, but also on the cellular environment. Single point mutations that decrease Aβ aggregation resulted in a more substantial activity increase of the secreted sensor, while the effect of Aβ40 was more pronounced on the cytoplasmic sensor (FIGS. 8A and B). Protein expression of the mutant Aβ chimeric reporters appeared to be comparable (FIG. 10). The E22G mutation, which is known to accelerate Aβ aggregation, decreased reporter activity (ratio Aβvar/Aβ42 wt <1), but this effect is only observed in the cytoplasmic environment (FIGS. 8A and 8B). We also performed in vitro aggregations of synthetic Aβ42 wt, Aβ40, Aβ42 F20E and Aβ42 E22G peptides and observed a correlation between the in vitro aggregation propensity of different Aβ variants with the activity of the cytoplasmic aggregation sensor (FIG. 8C).

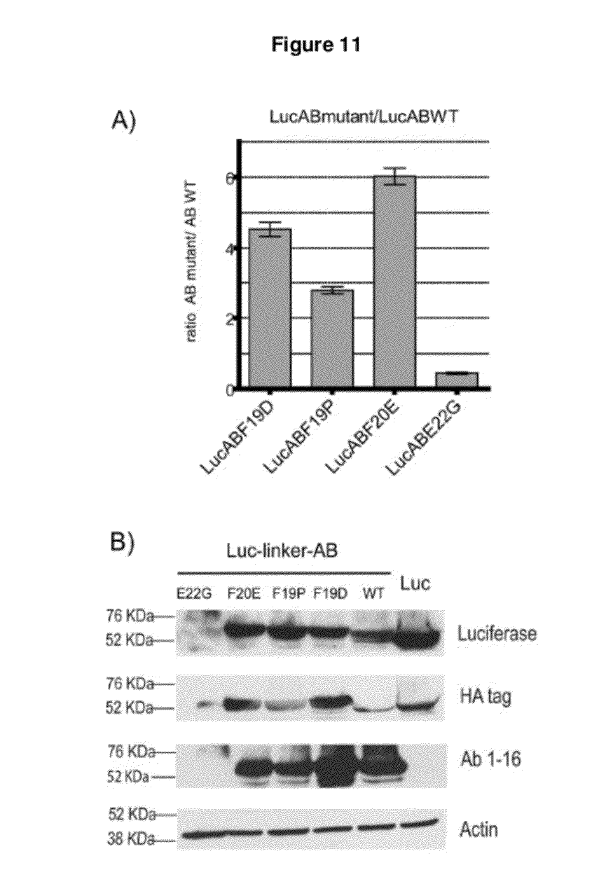

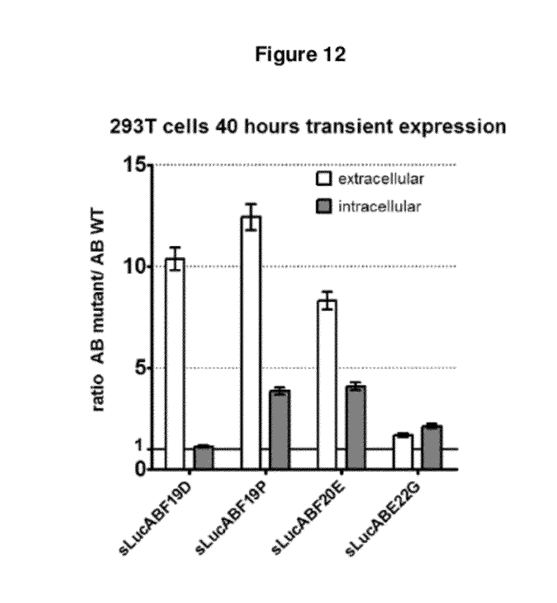

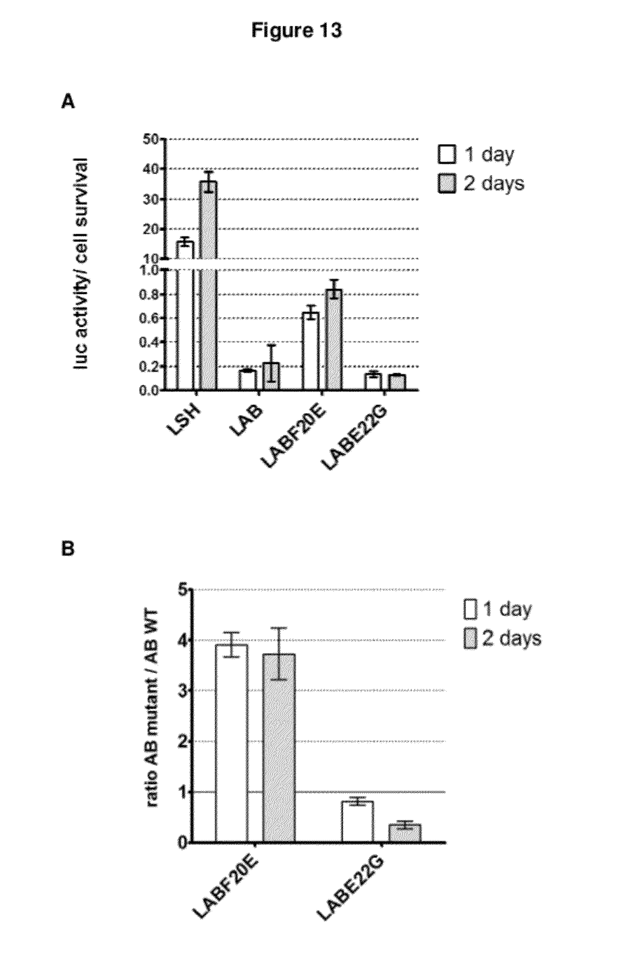

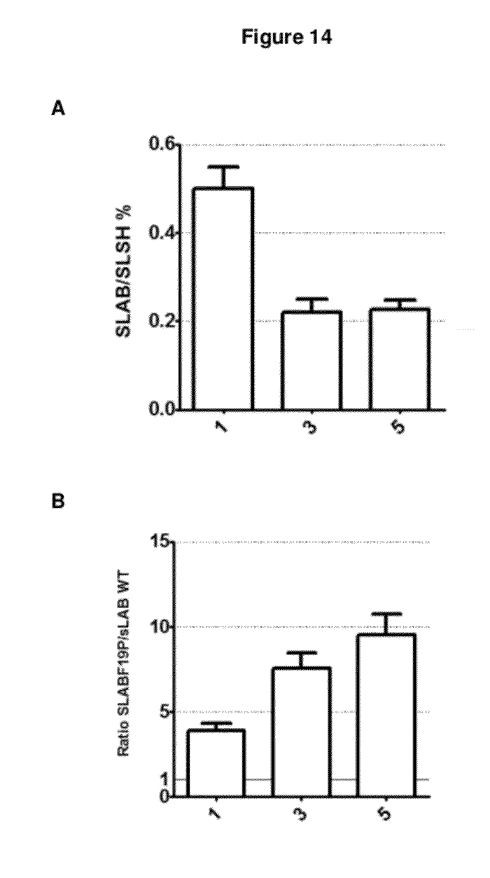

The results of additional assessments of the effect of mutations in Aβ on protein aggregation are provided in FIGS. 11-14.

### Example 5

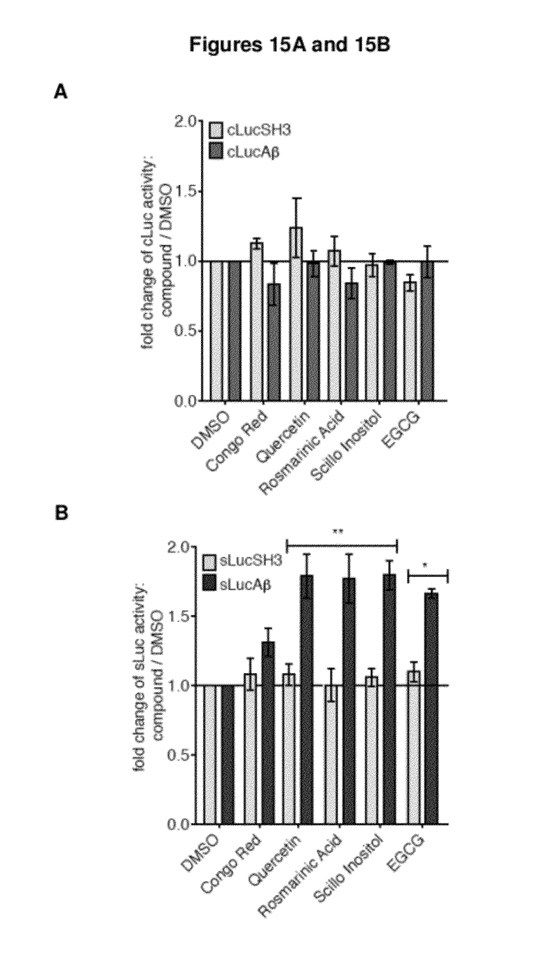

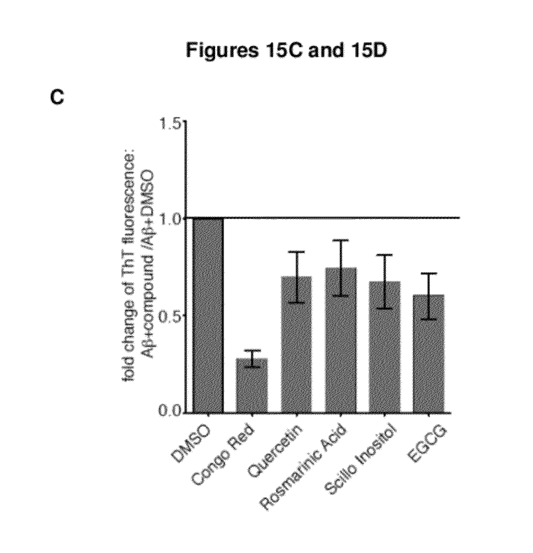

To evaluate whether not only intrinsic physico-chemical properties of the aggregating protein but also extrinsic factors could modulate the activity of the sensors, we assessed the effect of known small molecule aggregation inhibitors (FIGS. 15A and 15B) (M. Morell, et al. 2011, Ventura, Linking amyloid protein aggregation and yeast survival. Mol Biosyst; J. McLaurin et al., 2006, Cyclohexanehexyl inhibitors of Abeta aggregation prevent and reverse Alzheimer phenotype in a mouse model. Nat Med 12, 801; J. Bieschke et al., EGCG remodels mature alpha-synuclein and amyloid-beta fibrils and reduces cellular toxicity. Proc Natl Acad Sci USA 107, 7710). We observed that Quercetin, Rosmarinic acid, Scilloinositol and EGCG significantly increased activity of sLucAβ (FIG. 15B), but had no effect on cLucAβ (FIG. 15A). We also examined their effect on aggregation of synthetic Aβ42 peptide in vitro (FIG. 15C) and found that the ability of these compounds to reduce synthetic Aβ42 aggregation paralleled the rescue of sLucAβ activity, with one exception. Congo Red did not increase the activity of the secreted sensor, while it potently inhibited Aβ42 aggregation in vitro. One possible explanation for this discrepancy is that, despite its in vitro anti-aggregation properties, Congo Red is a nonselective binder and has unfavorable physico-chemical properties.

### Example 6

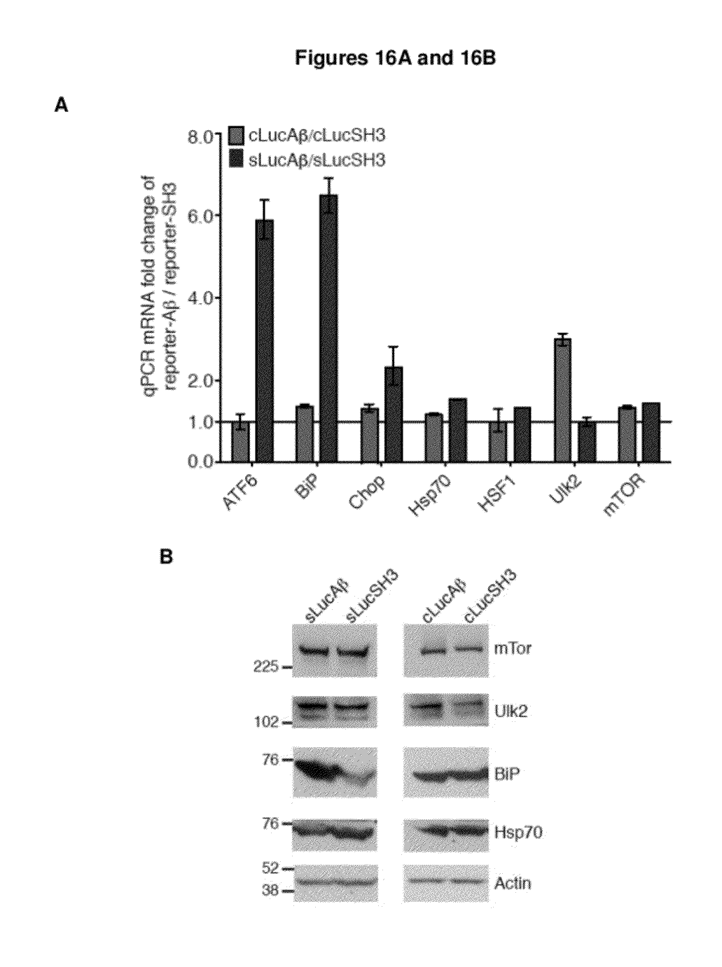

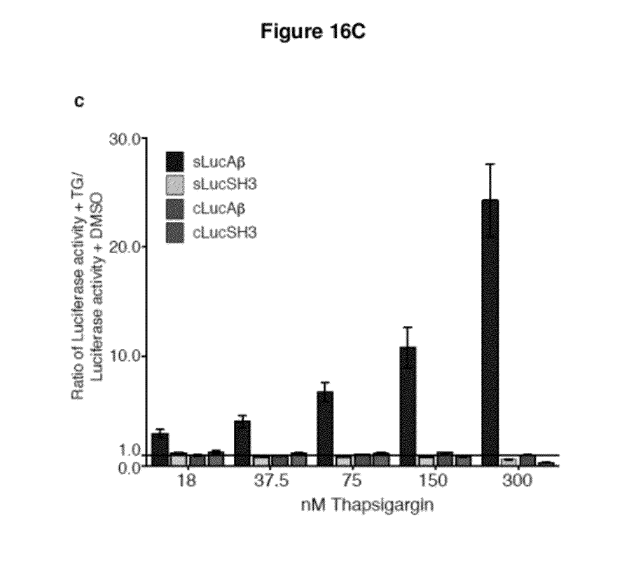

By taking advantage of the distinct cellular compartments in which the two bioluminescent sensors reside and aggregate, we investigated whether we could observe differences in the regulation of proteostasis signaling pathways following induction of either sLucAβ or cLucAβ. Signaling pathways that maintain protein homeostasis include: the ER stress and unfolded protein response (UPR); the heat shock response (HSR) and the autophagiclysosomal system (D. Ron, P. Walter, 2007, Signal integration in the endoplasmic reticulum unfolded protein response. Nat Rev Mol Cell Biol 8, 519; I. Shamovsky, E. Nudler, 2008, New insights into the mechanism of heat shock response activation. Cell Mol Life Sci 65, 855). We quantified mRNA expression levels of known ER stress, HSR and autophagy genes and found that, while aggregation within the secretory pathway resulted in upregulation of ER stress associated genes (ATF6, BiP and CHOP), aggregation in the cytoplasm led to increased expression of the autophagy regulator Ulk2 (D. F. Egan et al., Phosphorylation of ULK1 (hATG1) by AMP-activated protein kinase connects energy sensing to mitophagy. Science 331, 456). However, the HSR genes HSF1 and HSP70 were unchanged at 96 hours (FIG. 16A). Examination of BiP protein levels, which are increased in AD brains (J. J. Hoozemans et al., 2005, The unfolded protein response is activated in Alzheimer's disease. Acta Neuropathol 110, 165), confirmed its increase only following sLucAβ induction. Similarly, the protein expression levels of Ulk2 were increased only in cells expressing cLucAβ (FIG. 16B).

### Example 7

Thapsigargin (TG), an irreversible inhibitor of sarco(endo)plasmic reticulum Ca+2-ATPase, is known to induce the UPR and up-regulate expression of the ER-resident chaperone, BiP and other ER proteins (Ito D et al. 2004). TG treatment of inducible 293FlpInTrex cell lines expressing the aggregation sensors for 24 hours led to an increase of only sAβ activity in a TG dose-dependent manner (FIG. 16C). TG effect on cell viability was similar in all cell lines. This result indicates that the bioluminescent aggregation sensors can be used to quantitatively dissect the effect of modulating the proteostasis machinery in a specific subcellular compartment.

### Example 8

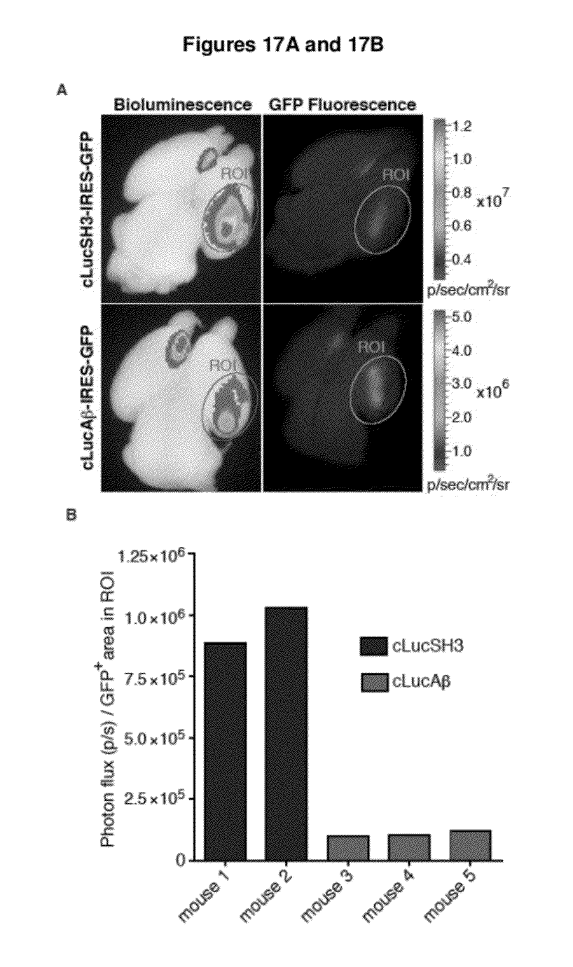

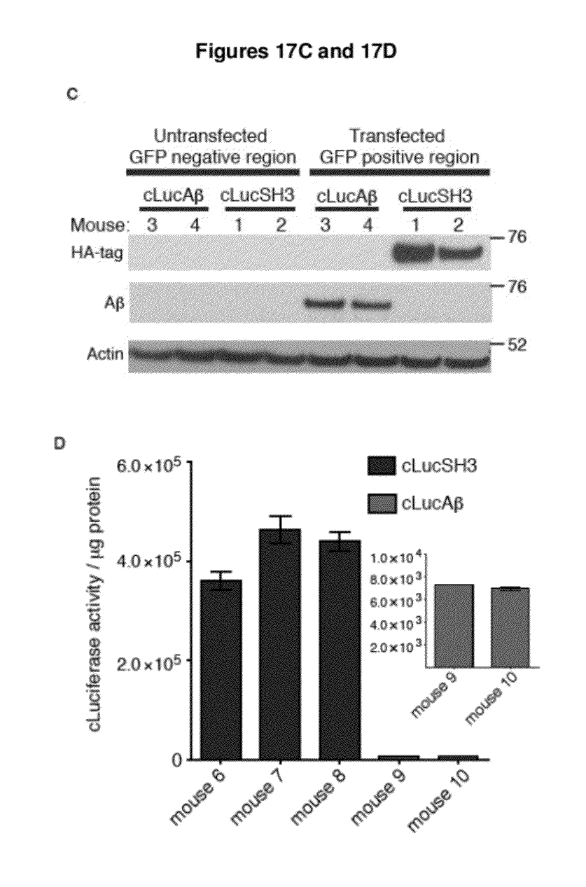

One of the advantages our sensors present is that luminescence can be used for quantitative, real time imaging of luciferase activity in live animals. Therefore, we examined whether the activity of the bioluminescent aggregation sensors in mouse brains mirrored the activity seen in tissue culture. We performed in utero electroporations of the cerebral cortex of E14.5 mouse embryos with bicistronic plasmids expressing either cLucAβ or cLucSH3 and nuclear GFP. The localization of GFP served to mark the transfected area and to normalize protein expression levels. We imaged and quantified bioluminescence emission of transfected cortex 3 days later (FIG. 17A). Fusion of Aβ42 to cLuc reduced photon flux in the transfected region by ˜10 fold compared to the control cLucSH3 reporter (FIG. 17B). We dissected the transfected area and found that protein expression paralleled the pattern seen in cultured cells (FIG. 17C and Table 4 below) and that the enzymatic activity of cLucAβ was ˜50 fold lower than cLucSH3 (FIG. 17D and Table 5 below). These results showed an excellent correlation between activity of the intracellular aggregation reporter in cell culture and in vivo, confirming that the bioluminescent sensors could make a valuable tool to test therapeutic strategies to block the aggregation of Aβ in vivo.

TABLE 4
Quantification of photon flux normalized by GFP
expression in the transfected cortical region (ROI)
of in utero electroporated mouse embryos.

| | Photon flux^{(a)}/GFP positive area^{(b)} |
|---|---|
| cLucSH3-mouse 1 | 8.85 10⁺⁵ |
| cLucSH3-mouse 2 | 1.03 10⁺⁶ |
| cLucAβ-mouse 3 | 1.01 10⁺⁵ |
| cLucAβ-mouse 4 | 1.05 10⁺⁵ |
| cLucAβ-mouse 3 | 1.14 10⁺⁵ |

^{(a)}Photon flux units: photons per second, p/s
^{(b)}GFP area units: pixels².

TABLE 5
In vitro luciferase activity of brain lysates
normalized by μg of protein lysate.

| | cLuciferase activity/μg protein lysate |
|---|---|
| cLucSH3-mouse 6 | 3.6 10⁺⁵ ± 1.8 10⁺⁴ |
| cLucSH3-mouse 7 | 4.6 10⁺⁵ ± 2.7 10⁺⁴ |
| cLucSH3-mouse 8 | 4.3 10⁺⁵ ± 1.8 10⁺⁴ |
| cLucAβ-mouse 9^{(a)} | 7.4 10⁺³ |
| cLucAβ-mouse 10 | 7.0 10⁺³ ± 1.5 10⁺² |

^{(a)}Dissected area was so small that only one measurement was done.

### Example 9

Tau constructs and mutants known in the art to promote aggregation (see, e.g. Chun, et al.(2007) J Neurochem. 103(6):2529-39) may be used in the aforementioned study may be fused to luciferase and assessed for their effect on enzymatic activity (FIG. 4). Optimized design in terms of position and linker obtained from studies with the Aβ42-luciferase constructs in employed. Aβ42 and Tau have been reported to be able to form a soluble complex that might facilitate Tau hyperphosphorylation, but they form separated insoluble deposits in the brain of AD patients. Co-expression of the Aβ42-luciferase and Tau-luciferase aggregation sensors may be used to probe if there is a synergistic effect on the aggregation of both proteins. Study of inclusion body formation using the techniques described before might help to elucidate if the two proteins can form deposits together.

### Example 10

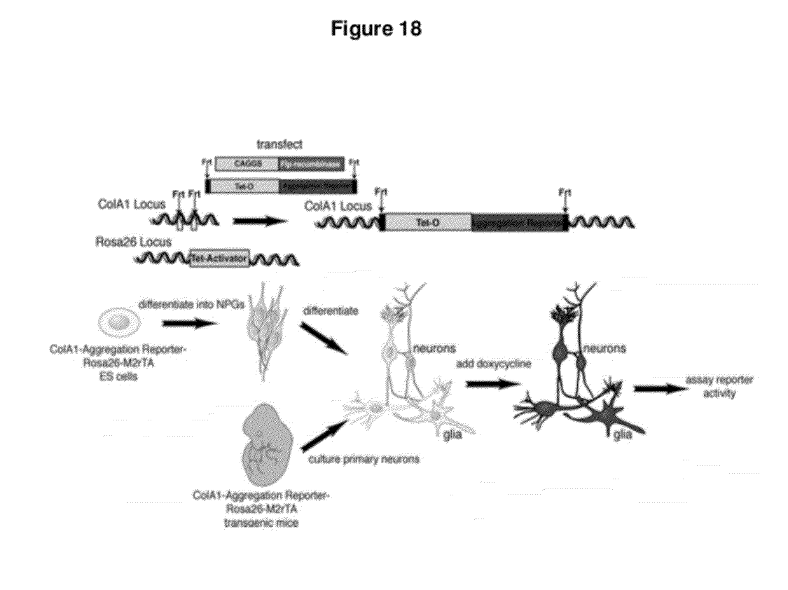

Generation of embryonic stem cell lines which express tetracycline-inducible aggregation reporters. To create a stable source of cells that can inducibly express the reporter and control proteins, the system to generate single copy transgenic mice or transgenic ES cells by site-specific integration is used (FIG. 18). This system is based on site specific recombination of a tetracycline-inducible transgene (Flp-in-TetO/transgene) into ES cells that were engineered to allow FLPe-recombinase mediated integration into the ColA1 locus. One advantage of this system is that the flp-in strategy avoids random integration of multiple copies. This ES cell line also expresses the M2rtTA-transactivator driven by the endogenous Rosa26 promoter and transgene expression can be induced by doxycycline treatment. The ES cell lines created can be used to either generate neurons by direct differentiation of ES cells into neurons in vitro, or to generate transgenic mice and use neurons cultured from the transgenic mice.

### Example 11

As an alternative strategy, the aggregation sensor is made based upon the split-firefly luciferase system (Paulmurugan, R. and S. S. Gambhir, Combinatorial library screening for developing an improved split firefly luciferase fragment-assisted complementation system for studying protein-protein interactions. Anal Chem, 2007. 79(6):2346-53). In this system, the luciferase enzyme is split in two fragments that, if allowed to fold properly when expressed, can reassemble into a functional enzyme when brought in proximity. If, on the other hand, these fragments are not allowed to fold properly when expressed, e.g. in such constructs in which the luciferase enzyme fragments separated by the Aβ42 peptide, they will not reassemble into a functional enzyme as a result of Aβ42 induced aggregation. See, for example, the “split luciferase” construct diagrammed in FIG. 2C.

Our results showed that the bioluminescent sensors are sensitive and versatile tools to probe protein aggregation properties in distinct subcellular compartments of neurons and to monitor protein aggregation in live brains and other tissues. We found that in vitro aggregation of Aβ variants correlated with aggregation in the cytosolic but not in the secretory pathway of hippocampal neurons and that Aβ aggregation in the two compartments triggered different responses of the proteostasis network. These findings illustrate that although in vitro experiments might give us a glimpse at the properties of aggregating proteins in the cytosolic environment, there are additional factors that influence protein aggregation in the secretory pathway. We also show an excellent correlation between activity of the intracellular aggregation reporter in cell culture and in vivo, and that similar design principles can be applied to monitor in vitro and in vivo aggregation of other pathogenic proteins.

Comparative measurements of aberrant protein aggregation in distinct cellular compartments open the exciting possibility of characterizing how cells derived from patients with neurodegenerative or other protein misfolding-associated diseases handle and respond to protein aggregation in different subcellular compartments, and of shedding light on molecular pathways contributing to the development of such diseases. This system will also enable execution of cell-based, chemical and genetic screens in the dynamic environment of mammalian neurons and validation of therapeutic strategies centered on aberrant protein aggregation in the brains or other tissues of live animals.

That which is claimed is:

1. An aggregation sensor, the aggregation sensor comprising:
a reporter polypeptide fused to one or more aggregating peptides.

2. The aggregation sensor according to claim 1, wherein the aggregation sensor is an intracellular aggregation sensor and the reporter polypeptide is an intracellular polypeptide.

3. The aggregation sensor according to claim 1, wherein the aggregation sensor is an extracellular sensor and the reporter polypeptide is a secreted polypeptide.

4. The aggregation sensor according to claim 1, wherein the aggregating peptide is selected from an Aβ peptide, Tau peptide, and an α-synuclein peptide.

5. The aggregation sensor according to claim 4, wherein the Aβ peptide is Aβ40 or Aβ42.

6. The aggregation sensor according to claim 5, wherein the Aβ peptide is a variant of Aβ40 or Aβ42 peptide.

7. The aggregation sensor according to claim 4, wherein the Tau peptide is ₂₄₄Tau₃₇₂.

8. The aggregation sensor according to claim 4, wherein the α-synuclein peptide is a variant comprising a substitution at residue 30.

9. The aggregation sensor according to claim 8, wherein the substitution is A30P.

10. The aggregation sensor according to claim 1, wherein the one or more aggregating peptides is fused to the N-terminus of the reporter polypeptide.

11. The aggregation sensor according to claim 1, wherein the one or more aggregating peptides is fused to the C-terminus of the reporter polypeptide.

12. The aggregation sensor according to claim 1, wherein the aggregation sensor comprises two or more aggregating peptides, wherein one or more aggregating peptides is fused to the N-terminus of the reporter polypeptide and one or more aggregating peptides is fused to the C-terminus of the reporter polypeptide.

13. A nucleic acid encoding an aggregating sensor, the nucleic acid comprising:
an expression cassette comprising sequence encoding a reporter polypeptide fused to one or more aggregating peptides.

14. The nucleic acid according to claim 13, wherein the sequence encoding reporter polypeptide fused to one or more aggregating peptides is operably linked to an inducible promoter.

15. The aggregation sensor according to claim 14, wherein the inducible promoter is a tetracycline promoter.

16. A method of screening a candidate agent for activity in reducing the aggregation of polypeptides, comprising:
contacting a cell comprising an aggregating sensor comprising a reporter polypeptide fused to one or more aggregating peptides with a candidate agent; and
comparing the activity of the reporter polypeptide to the activity of the reporter polypeptide in an aggregating sensor in a cell that was not contacted with the candidate agent;
wherein greater activity of the reporter polypeptide in the cell that was contacted with the candidate agent as compared to the reporter polypeptide in the cell that was not contacted with the candidate agent indicates that the candidate agent will reduce polypeptide aggregation.

17. The method according to claim 16, wherein the candidate agent is a small molecule, a nucleic acid, or a polypeptide.

18. The method according to claim 16, wherein the candidate agent that reduces the aggregation of polypeptides comprising the aggregating peptide will treat a disease associated with the abnormal accumulation of amyloid.

19. The method according to claim 18, wherein the disease associated with abnormal accumulation of amyloid is a neurodegenerative disease; Type 2 diabetes mellitus; medullary carcinoma of the thyroid; cardiac arrhythmias; solated atrial amyloidosis; atherosclerosis; rheumatoid arthritis; aortic medial amyloid; prolactinomas; familial amyloid polyneuropathy; hereditary non-neuropathic systemic amyloidosis; dialysis related amyloidosis; finnish amyloidosis; lattice corneal dystrophy; cerebral amyloid angiopathy; systemic AL amyloidosis; or Sporadic Inclusion Body Myositis.

20. The method according to claim 19, wherein the neurodegenerative disease is Alzheimer's disease, Parkinson's disease, transmissible spongiform encephalopathy, or Huntington's Disease.
